(19) United States
(12) Patent Application Publication
(10) Pub. No.: US 2026/0257193 A1
(43) Pub. Date: Sep. 3, 2026

(54) CARBON MOLECULAR MEMBRANE REACTOR SYSTEMS, AND METHODS FOR FABRICATION AND USE THEREOF

(71) Applicant: UNIVERSITY OF MARYLAND, COLLEGE PARK, College Park, MD (US)

(72) Inventors: Dongxia LIU, Clarksville, MD (US); Chen ZHANG, Laurel, MD (US); Lu LIU, Greenbelt, MD (US); Antara BHOWMICK, College Park, MD (US)

(21) Appl. No.: 18/993,196

(22) PCT Filed: Jul. 12, 2023
(86) PCT No.: PCT/US23/27433

§ 371 Date: Jan. 10, 2025

Related U.S. Application Data
(60) Provisional application No. 63/368,248, filed on Jul. 12, 2022.

(51) Int. Cl. B01J 19/24; C01B 3/26

(52) U.S. Cl. CPC B01J 19/2475; C01B 3/26; C01B 2203/041; C01B 2203/085; C01B 2203/107; C01B 2203/1076

(57) ABSTRACT

A reactant flow can be provided to a first volume of a catalytic membrane reactor. The catalytic membrane reactor can include at least one hollow fiber membrane and at least one catalyst. Each hollow fiber membrane can have an annular wall formed of carbon. The catalytic membrane reactor can be subjected to an elevated temperature, such that the reactant in the reactant flow is converted to one or more products and such that hydrogen permeates through the annular wall and is carried from a second volume of the catalytic membrane reactor by a sweep gas flow or vacuum.

### CROSS-REFERENCE TO RELATED APPLICATION(S)

The present application claims the benefit of U.S. Provisional Application No. 63/368,248, filed Jul. 12, 2022, entitled “Carbon Molecular Membrane Reactor,” which is hereby incorporated by reference herein in its entirety.

### STATEMENT REGARDING FEDERALLY SPONSORED RESEARCH

This invention was made with government support under CBET1928325 awarded by the National Science Foundation (NSF). The government has certain rights in the invention.

### FIELD

The present disclosure relates generally to reactors for chemical conversion, and more particularly, to catalytic membrane reactor systems and methods.

### BACKGROUND

Light alkenes have been used as chemical building blocks in the production of industrial products, such as polymers, oxygenates, and chemical intermediates. Non-oxidative dehydrogenation can yield a particular alkene and hydrogen (H₂) from the corresponding alkane. However, the endothermic nature of the non-oxidative dehydrogenation reaction requires high temperatures to attain attractive conversion for industrial applications. Such high temperatures can result in side reactions (e.g., cracking and coke formation) and/or catalyst deactivation due coking or sintering. Thus, catalyst regeneration via oxidative de-coking and re-reduction cycles is often necessary in conventional moving or fix-bed reactor systems. Moreover, conventional alkane dehydrogenation reactors are heated by fossil fuel firing, which can contribute to CO₂ emissions.

Membrane reactors (MRs) have the potential to overcome the thermodynamic limit by in-situ removal of by-products, resulting in increased product yields, lower reaction temperatures, and/or improved energy efficiency. However, the use of alkane dehydrogenation MRs in conventional systems has been limited by at least two major challenges: (1) the catalyst and membrane can experience accelerated coking and deactivation due to H₂ depletion, and (2) the inorganic or metallic membranes can be costly to fabricate at large scale. For example, conventional propane dehydrogenation (PDH) reactions can employ alumina supported chromium oxide (Cr₂O₃/Al₂O₃) or platinum-tin (Pt—Sn/Al₂O₃) catalysts operating at temperatures in range of about 600-650° C. While good propane conversion (e.g., ~35-50%) and C₃H₆ selectivity (e.g., ~80-90%) can be achieved, the catalysts in conventional systems need to be regenerated periodically (Cr₂O₃/Al₂O₃, every 7-15 minutes; Pt—Sn/Al₂O₃, every 7-10 days) to remove coke deposit and maintain activity. Process heat from direct fired furnaces or catalyst coke combustion can produce CO₂ emissions.

MRs using H₂-permeable palladium (Pd)-based metal or inorganic oxide (e.g., alumina, silica, or zeolite) membranes, in combination with PDH catalysts, have been pursued to yield high conversions at low reaction temperatures. However, these conventional Pd-based MRs suffer from rapid drop of conversion due to deactivation of membrane and catalyst materials. Although inorganic oxide MRs had better stability, propane conversion enhancement was low. To date, achieving high reactant conversion, high product yield, high catalyst durability, and low CO₂ emissions in non-oxidative alkane dehydrogenation remains a challenge.

Embodiments of the disclosed subject matter may address one or more of the above-noted problems and disadvantages, among other things.

### SUMMARY

Embodiments of the disclosed subject matter provide catalytic membrane reactors and reactor systems for converting a reactant into one or more products, for example, via non-oxidative dehydrogenation. In some embodiments, the reactant can comprise an alkane, and the product can comprise a corresponding alkene. In some embodiments, the membrane reactor comprises a hydrogen-permeable hollow fiber membrane formed of carbon. In some embodiments, at least part of the hollow fiber membrane may be porous (also referred to herein as a carbon molecular sieve (CMS)). In some embodiments, one or more catalysts can be provided in or adjacent to a reactant flow volume of the membrane reactor. For example, the one or more catalysts can comprise a siliceous zeolite supported metal catalyst (also referred to herein as a metal/zeolite catalyst).

In some embodiments, the use of the metal/zeolite catalyst can lower the reaction temperature for the non-oxidative alkane dehydrogenation. In some embodiments, the reaction temperature can be less than or equal to 1000° C., for example, in a range of 300-600° C. In some embodiments, the use of a carbon-based membrane can overcome a thermodynamic limit of the non-oxidative alkane dehydrogenation. By lowering the reaction temperature and overcoming the thermodynamic limit, some embodiments of the disclosed subject matter can achieve higher conversion (e.g., at least 30% for conversion of propane; at least 12% for conversion of ethane) and/or improved catalyst stability (e.g., at least 100 hours). In some embodiments, the use of a carbon-based membrane can avoid, or at least reduce, greenhouse gas emissions (e.g., CO₂) by applying an electrical current to the membrane to cause Joule heating thereof in order to provide the elevated temperature for the reaction.

In one or more embodiments, a method can comprise providing a reactant flow in a first volume of a catalytic membrane reactor, and simultaneously providing a sweep gas flow in a second volume of the catalytic membrane reactor. The catalytic membrane reactor can comprise at least one hollow fiber membrane and at least one catalyst. Each hollow fiber membrane can comprise an annular wall formed of carbon. Each annular wall can separate an interior volume from a surrounding outer volume. In some embodiments, the first volume comprises the surrounding outer volume, and the second volume comprises each interior volume. Alternatively, in some embodiments, the first volume comprises each interior volume, and the second volume comprises the surrounding outer volume.

The method can further comprise subjecting the catalytic membrane reactor to an elevated temperature less than 1000° C. (e.g., in a range of 100-1000° C., inclusive) such that one or more reactants in the reactant flow are converted to one or more products and such that hydrogen permeates through the annular wall and is carried from the second volume by the sweep gas flow or vacuum. In some embodiments, the subjecting to the temperature can be performed at a same time as (e.g., concurrent with) the providing of the reactant and sweep gas flows in the catalytic membrane reactor.

In one or more embodiments, a system can comprise a catalytic membrane reactor. The catalytic membrane reactor can comprise at least one hollow fiber membrane and at least one catalyst in or adjacent to a reactant flow volume. Each hollow fiber membrane can comprise an annular wall formed of carbon. Each annular wall can separate an interior volume from a surrounding outer volume. In some embodiments, the reactant flow volume comprises the surrounding outer volume, and the sweep flow volume comprises each interior volume. Alternatively, in some embodiments, the reactant flow volume comprises each interior volume, and the sweep flow volume comprises the surrounding outer volume. The catalytic membrane reactor can be configured such that a reactant flow is provided to the at least one catalyst via the reactant flow volume, a product flow exits from the reactant flow volume, and a permeate flow exits from the sweep flow volume.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Embodiments will hereinafter be described with reference to the accompanying drawings, which have not necessarily been drawn to scale. Where applicable, some elements may be simplified or otherwise not illustrated in order to assist in the illustration and description of underlying features. Throughout the figures, like reference numerals denote like elements.

FIG. 1A is a simplified cross-sectional view illustrating aspects of a catalytic membrane reactor, according to one or more embodiments of the disclosed subject matter.

FIG. 1B is a simplified cross-sectional view illustrating aspects of a catalytic membrane reactor with two-region membrane, according to one or more embodiments of the disclosed subject matter.

FIGS. 2A-2B are simplified cross-sectional and axial views, respectively, of a catalytic membrane reactor employing a single hollow fiber membrane, according to one or more embodiments of the disclosed subject matter.

FIGS. 2C-2D are simplified cross-sectional and axial views, respectively, of another catalytic membrane reactor employing a single hollow fiber membrane, according to one or more embodiments of the disclosed subject matter.

FIGS. 2E-2F are simplified cross-sectional and axial views, respectively, of a catalytic membrane reactor employing multiple hollow fiber membranes, according to one or more embodiments of the disclosed subject matter.

FIGS. 2G-2H are simplified cross-sectional and axial views, respectively of another catalytic membrane reactor having a catalyst within a porous region of a hollow fiber membrane, according to one or more embodiments of the disclosed subject matter.

FIG. 3A is a simplified cutaway view illustrating aspects of a catalytic membrane reactor with multiple hollow fiber membranes for alkane dehydrogenation, according to one or more embodiments of the disclosed subject matter.

FIG. 3B shows scanning electron microscopy (SEM) images of an asymmetric hollow fiber membrane, according to one or more embodiments of the disclosed subject matter.

FIG. 4A is a simplified schematic diagram illustrating aspects of a system with a catalytic membrane reactor heated by a furnace, according to one or more embodiments of the disclosed subject matter.

FIG. 4B is a simplified schematic diagram illustrating aspects of another system with a Joule-heated catalytic membrane reactor, according to one or more embodiments of the disclosed subject matter.

FIG. 4C is a simplified schematic diagram illustrating aspects of another system with a solar-heated catalytic membrane reactor, according to one or more embodiments of the disclosed subject matter.

FIG. 5A illustrates a process flow diagram for a catalytic membrane reactor method, according to one or more embodiments of the disclosed subject matter.

FIG. 5B depicts a generalized example of a computing environment in which the disclosed technologies may be implemented.

FIGS. 6A-6B are graphs of conversion (divided by thermodynamic equilibrium conversion) versus temperature for various catalysts and for various membrane reactors with catalysts, respectively, for propane dehydrogenation.

FIG. 6C is a graph of deactivation rate versus conversion (divided by thermodynamic equilibrium conversion) for various membrane reactors for propane dehydrogenation.

FIGS. 6D-6E are graphs showing propane conversion, propylene yield, and propylene selectivity as a function of time for carbon molecular sieve (CMS) membrane reactors (MR) and a packed-bed reactor (PBR). PBR conditions included a reaction temperature of 450° C., a weight hourly space velocity (WHSV) of 0.35 h⁻¹, and 50% C₃H₈ balanced with argon (Ar). MR conditions were the same as PBR and further included 100 mL/min sweep gas flow and five CMS hollow fibers in the reactor.

FIG. 7A is a graph of permeances and separation factors in a membrane reactor employing CMS hollow fibers. Testing conditions include a sweep gas flow rate of 100 mL/min, a reaction temperature of 450° C., a WHSV of 1.73 h⁻¹ (50% C₃H₈/50% Ar), and five CMS hollow fibers in the reactor.

FIG. 7B is a graph of propane conversion and propylene selectivity over Pt—Zn/S1 catalyst in the PBR and MR at different reaction temperatures. Testing conditions include a sweep gas flow rate of 100 mL/min, a WHSV of 1.73 h⁻¹ (50% C₃H₈/50% Ar), and five CMS hollow fibers in the reactor.

FIG. 7C is a graph of propane conversion and propylene selectivity over Pt—Zn/S1 catalyst in the MR for different numbers of CMS hollow fibers in the reactor. Testing conditions include a sweep gas flow rate of 100 mL/min, a WHSV of 1.73 h⁻¹ (50% C₃H₈/50% Ar), and a reaction temperature of 450° C.

FIG. 7D is a graph of propane conversion and propylene selectivity over Pt—Zn/S1 catalyst in the MR for different WHSV values. Testing conditions include a sweep gas flow rate of 100 mL/min, a reaction temperature of 450° C., and five CMS hollow fibers in the reactor.

FIG. 7E is a graph of propane conversion and propylene selectivity over Pt—Zn/S1 catalyst in the MR for different propane feed partial pressures. Testing conditions include a sweep gas flow rate of 100 mL/min, a WHSV of 0.35 h⁻¹ (50% C₃H₈/50% Ar), a reaction temperature of 450° C., and five CMS hollow fibers in the reactor.

FIG. 7F is a graph of propane conversion and propylene selectivity over Pt—Zn/S1 catalyst in the MR for different sweep gas flow rates. Testing conditions include a WHSV of 1.73 h⁻¹ (50% C₃H₈/50% Ar), a reaction temperature of 450° C., and five CMS hollow fibers in the reactor.

FIG. 8A is a graph of H₂ permeance and H₂/C₂H₆ separation factor of a CMS hollow fiber membrane versus permeation temperature.

FIG. 8B is a graph of ethane conversion and ethylene selectivity over a Co/deAl-BEA catalyst in the PBR and MR at different reaction temperatures. PBR conditions included a WHSV of 1.43 h⁻¹, and 50% C₂H₆ balanced with Ar. MR conditions were the same as PBR and further included 100 mL/min sweep gas flow and five CMS hollow fibers in the reactor.

FIGS. 8C-8D are graphs showing ethane conversion, ethylene yield, and ethylene selectivity as a function of time for the MR and PBR. PBR conditions included a reaction temperature of 500° C., a WHSV of 0.29 h⁻¹, and 50% C₂H₆ balanced with Ar. MR conditions were the same as PBR and further included 100 mL/min sweep gas flow and five CMS hollow fibers in the reactor.

FIGS. 8E-8F are graphs of conversion (divided by thermodynamic equilibrium conversion) versus temperature for various catalysts and for various membrane reactors with catalysts, respectively, for ethane dehydrogenation.

FIG. 8G is a graph of deactivation rate versus conversion (divided by thermodynamic equilibrium conversion) for various membrane reactors for ethane dehydrogenation.

FIG. 9A is a graph of temperature versus power applied to Joule heat a CMS hollow fiber (60 mm in length).

FIG. 9B is a graph of measured proposed conversion and propylene selectivity in a Joule-heated CMS MR versus average reaction temperature. The average reaction temperature was obtained by averaging the 2D axisymmetric temperatures profiles. MR conditions included a 10 mL/min sweep gas flow, a WHSV of 1.33 h⁻¹, 50% C₃H₈ balanced with Ar, and 1 CMS hollow fiber in the reactor.

### DETAILED DESCRIPTION

### General Considerations

For purposes of this description, certain aspects, advantages, and novel features of the embodiments of this disclosure are described herein. The disclosed methods and systems should not be construed as being limiting in any way. Instead, the present disclosure is directed toward all novel and nonobvious features and aspects of the various disclosed embodiments, alone and in various combinations and sub-combinations with one another. The methods and systems are not limited to any specific aspect or feature or combination thereof, nor do the disclosed embodiments require that any one or more specific advantages be present, or problems be solved. The technologies from any embodiment or example can be combined with the technologies described in any one or more of the other embodiments or examples. In view of the many possible embodiments to which the principles of the disclosed technology may be applied, it should be recognized that the illustrated embodiments are exemplary only and should not be taken as limiting the scope of the disclosed technology.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one skilled in the art.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person skilled in the art to have a more definitive construction, the numerical parameters set forth are approximations that may depend on the desired properties sought and/or limits of detection under standard test conditions/methods, as known to those skilled in the art. When directly and explicitly distinguishing embodiments from discussed prior art, the embodiment numbers are not approximates unless the word “about,” “substantially,” or “approximately” is recited. Whenever “substantially,” “approximately,” “about,” or similar language is explicitly used in combination with a specific value, variations up to and including 10% of that value are intended, unless explicitly stated otherwise.

Directions and other relative references may be used to facilitate discussion of the drawings and principles herein but are not intended to be limiting. For example, certain terms may be used such as “inner,” “outer,” “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part, and the object remains the same.

As used herein, “comprising” means “including,” and the singular forms “a” or “an” or “the” include plural references unless the context clearly dictates otherwise. The term “or” refers to a single element of stated alternative elements or a combination of two or more elements unless the context clearly indicates otherwise.

Although there are alternatives for various components, parameters, operating conditions, etc. set forth herein, that does not mean that those alternatives are necessarily equivalent and/or perform equally well. Nor does it mean that the alternatives are listed in a preferred order, unless stated otherwise. Unless stated otherwise, any of the groups defined below can be substituted or unsubstituted.

Unless explained otherwise, all technical and scientific terms used herein have the same meaning as commonly understood to one skilled in the art to which this disclosure belongs.

Although methods and materials similar or equivalent to those described herein can be used in the practice or testing of the present disclosure, suitable methods and materials are described below. The materials, methods, and examples are illustrative only and not intended to be limiting. Features of the presently disclosed subject matter will be apparent from the following detailed description and the appended claims.

### Overview of Terms

The following are provided to facilitate the description of various aspects of the disclosed subject matter and to guide those skilled in the art in the practice of the disclosed subject matter.

Catalytic Membrane Reactor: A device having one or more membranes and one or more catalysts. The catalyst(s) can catalyze conversion of a reactant into one or more products, for example, via a dehydrogenation reaction. The membrane(s) can allow selective passage of atoms or molecules therethrough, for example, to remove or isolate one or more products. In some embodiments, the membrane(s) can be a carbon molecular sieve, and/or the catalyst can be a siliceous zeolite confined metal (M/zeolite, where M is a metal). In some embodiments, the catalytic membrane reactor can be configured as a moving bed membrane reactor, a fixed bed membrane reactor, a fluidized bed membrane reactor, or a packed bed membrane reactor.

Carbon molecular sieve (CMS): A circumferentially-extending (e.g., annular) wall formed of carbon and having at least a porous region. For example, pores in the porous region can have a size (e.g., maximum cross-sectional dimension, such as diameter) in a range of 0.3-0.7 nm, inclusive. In some embodiments, the wall can be formed by pyrolysis of an organic precursor, such as, but not limited to a polymer, coke, coal, and biomass. In some embodiments, the circumferentially-extending wall can also have a dense region (e.g., a hydrogen separation layer), for example, adjacent to the porous region (e.g., a support layer). In some embodiments, the circumferentially-extending wall forms a hollow fiber membrane (e.g., having an outer diameter less than or equal to 0.5 mm, for example, about 300-315 μm, and/or an inner diameter less than or equal to 200 μm, for example about 150 μm), the porous region can be a radially-innermost annular region of the wall, and the dense region can be a radially-outermost annular region of the wall. In some embodiments, the dense region can have a density greater than that of the porous region, and/or pores in the dense region can have a size (e.g., maximum cross-sectional dimension, such as diameter) less than 0.3 nm. In some embodiments, one or more catalysts can reside in the porous region.

Lewis acidic center: A chemical species (or the portion thereof acting as an acid) that operates as an acid by accepting a pair of electrons. For example, the chemical species can be a molecule or ion that have an incomplete octet of electrons.

Brønsted acidic center: A chemical species (or the portion thereof acting as an acid) that operates as an acid by donating a proton (e.g., hydrogen ion, H⁺). For example, the chemical species can be a molecule or ion that contains a hydrogen atom bonded to a highly electronegative element, such as oxygen or nitrogen.

Inert Gas: One or more gases that do not undergo a chemical reaction when subjected to the reaction temperature. In some embodiments, the inert gas is selected from the group consisting of nitrogen, argon, helium, neon, krypton, xenon, radon, and oganesson.

### Introduction

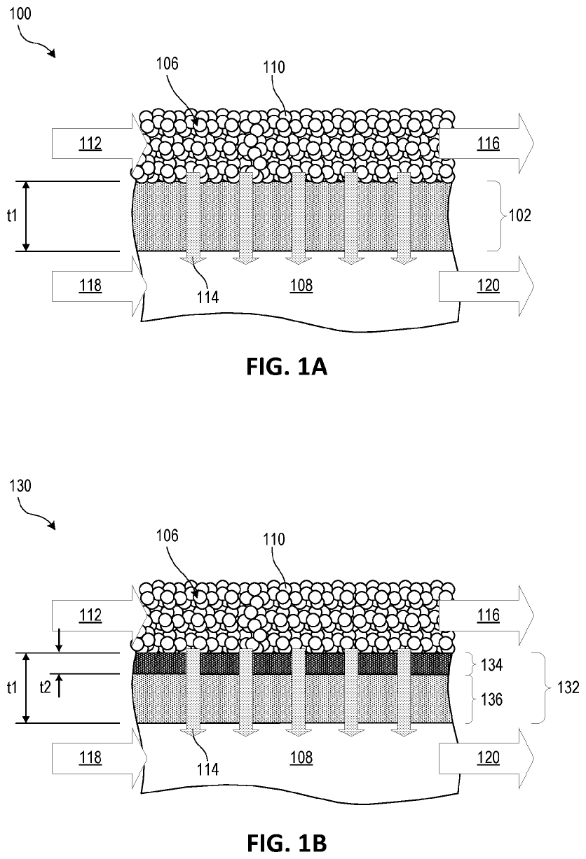

Disclosed herein are catalytic membrane reactors having a carbon molecular sieve (CMS) membrane and a catalyst, systems including such catalytic membrane reactors, and methods for fabrication and use of such catalytic membrane reactors. In some embodiments, the catalytic membrane reactor can be used to perform a direct non-oxidative conversion reaction, such as an alkane dehydrogenation reaction. As shown in FIG. 1A, a catalytic membrane reactor 100 can have a CMS membrane 102, a first volume 106 (also referred to herein as reaction volume or reactant flow volume), a second volume 108 (also referred to herein sweep flow volume or permeate volume), and one or more catalysts 110 provided in first volume 106 (e.g., disposed in, flowed into, and/or flowed through) or adjacent to first volume 106 (e.g., embedded within and/or supported by a portion of CMS membrane 102).

In some embodiments, for example, when the catalytic membrane reactor 100 is used for alkane dehydrogenation, catalyst(s) 110 can comprise a siliceous zeolite confined metal (M/zeolite) catalyst (e.g., MFI zeolite, MWW zeolite, FER zeolite, FAU zeolite, BEA zeolite, MOR zeolite). The metal in the catalyst can be Pt, Zn, Sn, Cu, Co, Pd, Fe, Ni, Ru, Rh, Ir, Y, Bi, Zr, Cr, Cs, Li, Na, K, Ca, Ba, or any combination thereof. In some embodiments, the concentration of metal in the catalyst can be in a range of 0.001 wt % to 30 wt %, inclusive. The absence of Lewis or Brønsted acidic centers in the M/zeolite can eliminate, or at least reduce, acid-catalyzed side reactions (such as cracking and oligomerization) that could otherwise lead to low alkene selectivity and catalyst deactivation due to coke deposition. In addition, the small metal clusters (e.g., single metal atoms, sub-nanometer-size clusters of metal, or nanometer-size clusters of metal) in the M/zeolite can be effective for alkane activation and suppression of side-reactions involving hydrogenolysis (C—C cleavage) and/or coke formation (C—C coupling) that could otherwise occur with geometrically complex and large ensembles of metal atoms. The confinement of zeolite micropores can prevent, or at least reduce, metal site sintering and consequent deactivation of the catalyst.

A feed gas flow 112 (also referred to herein as a reactant flow) including one or more reactants (e.g., alkane, such as propane or ethane) can be provided to (e.g., flowed into and/or through, for example, via positive pressure applied to an inlet of the reactor 100 and/or vacuum applied to an outlet of the reactor 100) the first volume 106. In some embodiments, a sweep gas flow 118 (e.g., an inert gas, such as Ar) can be provided to (e.g., flowed into and/or through, for example, via positive pressure applied to another inlet of the reactor 100 and/or vacuum applied to another outlet of the reactor 100) the second volume 108. The sweep gas flow 118 can carry off the permeate (e.g., hydrogen gas, H₂) from the second volume 108, for example, as permeate flow 120. Alternatively, in some embodiments, permeate flow 120 can be generated by applying a vacuum to an outlet of the reactor 100 without providing any sweep gas.

The first volume 106 can be separated from the second volume 108 by CMS membrane 102, which can be constructed to transport H₂ therethrough (e.g., as H₂ transport 114) at elevated temperatures (e.g., at least 100° C., such as at least 300° C.). The conversion of a reactant to one or more products can occur within the first volume 106, and the products and any unconverted reactants removed via an outlet flow 116 (also referred to herein as a product flow). The removal of H2 from the first volume 106 (where the conversion reaction takes place) can improve the conversion efficiency for the reactant (e.g., up to 40%).

In some embodiments, the CMS membrane 102 can have a substantially uniform composition and/or porosity across its thickness (e.g., a symmetric configuration), as shown in FIG. 1A. For example, the CMS membrane 102 can be a porous structure (e.g., having pore sizes that allow H₂ to pass through the membrane, but which prevent, or at least inhibit, passage of reactants and/or other products therethrough). In some embodiments, the thickness, t₁, of the membrane can be less than 200 μm, for example, about less than 100 μm (e.g., about 75 μm). Alternatively, in some embodiments, a CMS membrane 132 can have regions 134, 136 of different composition and/or porosity (e.g., an asymmetric configuration), as shown by the catalytic membrane reactor 130 of FIG. 1B. For example, a first region 136 of CMS membrane 132 can have a porosity greater than that of a second region 134 of CMS membrane 132, and/or the first region 136 can have a density less than that of the second region 134. In some embodiments, the second region 134 can be disposed between the first region 136 and the first volume 106 (e.g., with respect to a thickness direction and/or radial direction of the reactor 130), and the first region 136 can be disposed between the second region 134 and the second volume 108 (e.g., with respect to the thickness and/or radial direction). For example, the second region 134 can be disposed adjacent to the first volume 106, and the first region 136 can be disposed adjacent to the second volume 108.

In some embodiments, the second region 134 can be a separate layer formed on or over the first region 136. In such embodiments, the second region 134 can be formed of a same material as the first region 136 or a different material from the first region 136. Alternatively, in some embodiments, the second region 134 can be a portion of a single continuous layer treated (e.g., with a silane, such as vinyltrimethoxysilane (VTMS)) to have a higher density and/or lower porosity, and the first region 136 can be an untreated portion of the single continuous layer. In some embodiments, both the first and second regions are formed of carbon (e.g., consist essentially of carbon). In some embodiments, a thickness t₂ of the second region 134 can be no more than a thickness (e.g., t₁-t₂) of the first region 136. For example, a thickness t₂ of the second region 134 can be less than or equal to 5 μm (e.g., about 4.5 μm), and/or a thickness t₁ of the CMS membrane can be less than 150 μm (e.g., about 85 μm).

In the illustrated examples of FIGS. 1A-1B, the catalyst 110 is disposed in the reactant flow volume 106 and separate from the CMS membranes 102, 132. Alternatively or additionally, in some embodiments, the catalyst 110 can be disposed within and/or supported by the CMS membrane. For example, the catalyst 110 can be formed in, embedded within, or infiltrated into a porous region 136 of CMS membrane 132 in FIG. 1B, first volume 106 can operate as the sweep flow volume, and second volume 108 can operate as the reactant flow volume (e.g., with the conversion reaction occurring within the porous region 136).

### Catalytic Membrane Reactors

In some embodiments, each CMS membrane in a catalytic membrane reactor can be in the form of a hollow carbon tube or fiber, for example, having a circumferentially-extending (e.g., annular) wall surrounding an open core (also referred to herein as bore). The circumferentially-extending wall of the CMS membrane can serve as a thin separation layer that allows rapid H₂ removal from the reaction mixture, thereby up-shifting the reaction equilibrium to enable high conversion of reactant to products but at lower reaction temperatures than required in conventional systems. For example, in some embodiments, the catalytic membrane reactor can operate at a reaction temperature less than or equal to 1000° C. (e.g., in a range of 300-600 ° C., inclusive). In addition, in some embodiments, the relatively small diameter of the hollow carbon fiber (e.g., ≤500 μm) can offer significantly higher packing densities than conventional tubular or monolithic inorganic membranes, and therefore be more suitable for large-scale applications.

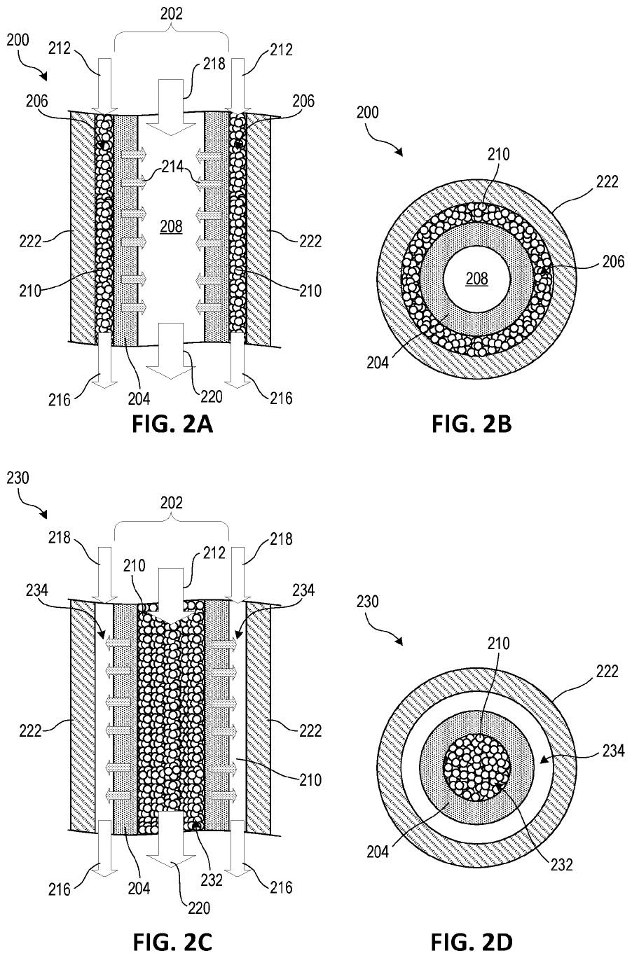

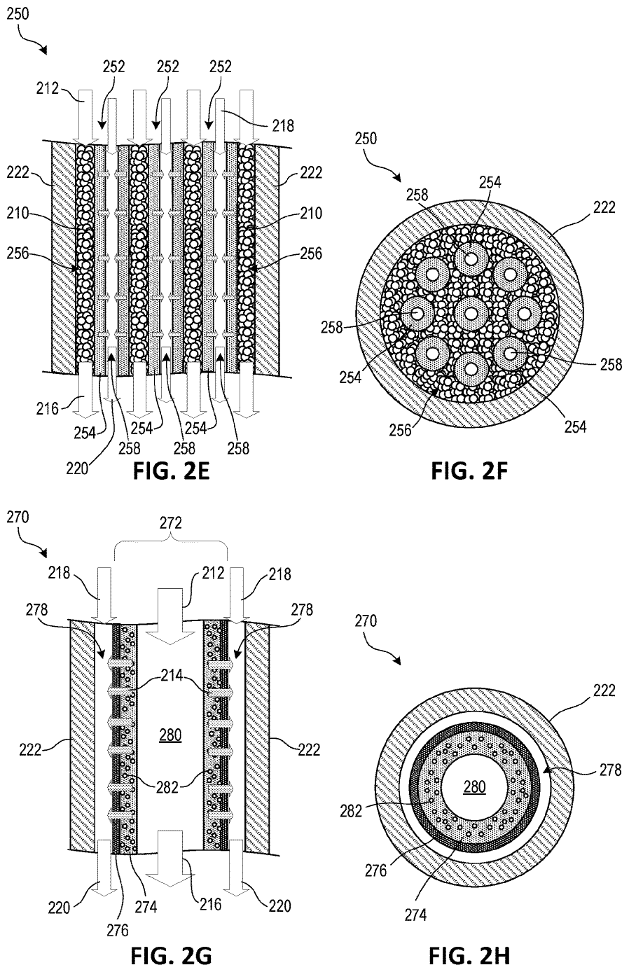

In some embodiments, a catalytic membrane reactor 200 can have a hollow fiber membrane 202 disposed within a shell or housing 222, for example, as shown in FIGS. 2A-2B. In the illustrated example, the hollow fiber membrane 202 has an annular wall 204 that separates an interior volume 208 from a surrounding outer volume 206 (e.g., an annular space defined between facing surfaces of the housing 222 and the wall 204). In the illustrated example, one or more catalysts 210 are provided in (e.g., disposed in, flowed into, and/or flowed through) the surrounding outer volume 206. As discussed above and elsewhere herein, the annular wall 204 can be formed of carbon (e.g., consisting essentially of carbon) and can have a symmetric or asymmetric (e.g., dual annular regions) configuration.

In operation, a feed gas flow 212 can comprise one or more reactants and can be provided to the outer volume 206 (e.g., by flowing into an inlet and/or applying a vacuum to an outlet of the reactor 200). The reactants in the feed gas flow 212 can interact with the catalyst at an elevated temperature and can be converted to hydrogen gas and one or more additional products. The hydrogen gas 214 can be transported radially inward, from the outer volume 206 through the annular wall 204 of the membrane 202 into the interior volume 208. Within the interior volume 208, a sweep gas flow 218 can carry the transported hydrogen gas out of the reactor 200 as a permeate flow 220 (e.g., comprising hydrogen gas and an inert sweep gas). In some embodiments, a vacuum can be applied to the interior volume 208 so as to generate permeate flow 220 with or without use of a sweep gas. The products and/or unconverted reactants remaining in outer volume 206 can form a product flow 216 that can be transported out of the catalytic membrane reactor 200 for separation (e.g., to isolate the reactants from the products), re-processing (e.g., to redirect the reactants back to an inlet of the catalytic membrane reactor to serve as or combine with reactant flow 212), storage (e.g., for transport or later use of the products), and/or use.

In the illustrated example of FIGS. 2A-2B, the outer volume 206 is provided with the catalyst 210 and is used as the reaction volume, and the interior volume 208 is used as the sweep volume. However, in some embodiments, the reaction can instead occur within the interior volume of the hollow fiber membrane. For example, FIGS. 2C-2D illustrate another catalytic membrane reactor 230 with a hollow fiber membrane 202 disposed within housing 222. Similar to the configuration of reactor 200 in FIGS. 2A-2B, the hollow fiber membrane 202 has an annular wall 204 that separates an interior volume from surrounding outer volume. However, in the illustrated example of FIGS. 2C-2D, the one or more catalysts 210 are provided in the interior volume, such that the interior volume acts as the reactant flow volume 232 and the surrounding outer volume acts as the sweep volume 234. In operation of catalytic membrane reactor 230, the feed gas flow 212 can be provided to the interior volume 232, the sweep gas flow 218 (when used) can be provided to the surrounding outer volume 234, and the hydrogen gas 214 can be transported radially outward through the annular wall 204 of membrane 202.

In the illustrated examples of FIGS. 2A-2D, the catalyst is disposed within a reaction volume of the catalytic membrane reactor and separate from the hollow fiber membrane 202.

However, in some embodiments, the catalyst can be integrated with and/or supported by the hollow fiber membrane. For example, FIGS. 2G-2H illustrate another catalytic membrane reactor 270 with a hollow fiber membrane 272 disposed within housing 222. In the illustrated example, The hollow fiber membrane 272 has an asymmetric annular wall that separates an interior volume 280 from a surrounding outer volume 278 (e.g., an annular space defined between facing surfaces of the housing 222 and the wall). The asymmetric annular wall can have a first porous region 274 (e.g., a radially-inner annular layer) and a second dense region 276 (e.g., a radially-outer annular layer), and the catalyst 282 can reside within the first porous region 274. As discussed above and elsewhere herein, the first region 274 and second region 276 of the annular wall can be formed of carbon (e.g., consisting essentially of carbon).

In operation, a feed gas flow 212 can comprise one or more reactants and can be provided to the interior volume 280 (e.g., by flowing into an inlet and/or applying a vacuum to an outlet of the reactor 270). The reactants in the feed gas flow 212 can interact with the catalysts 282 residing within the porous region 274 and can be converted to hydrogen gas and one or more additional products. The hydrogen gas 214 can be transported radially outward, from the interior volume 280 through the second region 276 of the annular wall of the membrane 272 into the outer volume 278. Within the outer volume 278, a sweep gas flow 218 can carry the transported hydrogen out of the reactor 270 as permeate flow 220. In some embodiments, a vacuum can be applied to the outer volume 278 so as to generate permeate flow 220 with or without use of a sweep gas. As with the above described examples, the products and/or unconverted reactants remaining in interior volume 280 can form a product flow 216 that can be transported out of the catalytic membrane reactor 270 for separation, re-processing, storage, and/or use.

Although FIGS. 2A-2D and 2G-2H show a single hollow fiber membrane within housing 222, embodiments of the disclosed subject matter are not limited thereto. Rather, according to one or more contemplated embodiments, any number of hollow fiber membranes (e.g., 2 or more) can be provided. In some embodiments, the provision of multiple hollow fiber membranes can achieve higher reactant conversion than use of a single hollow fiber membrane. For example, FIGS. 2E-2F illustrate another catalytic membrane reactor 250 with multiple hollow fiber membranes 252 disposed within housing 222. In some embodiments, the hollow fiber membranes 252 can be substantially identical, each with an annular wall 254 that separates a respective interior volume 258 from a common surrounding outer volume 256 (e.g., the space between radially-outer surfaces of walls 254 and radially-inner surface of housing 222). For example, each annular wall can be formed of carbon (e.g., consisting essentially of carbon) and can have a symmetric or asymmetric (e.g., dual annular regions) configuration. In the illustrated example, one or more catalysts 210 are provided in (e.g., disposed in, flowed into, and/or flowed through) common outer volume 256.

In operation, feed gas flow 212 (with one or more reactants) can be provided to the outer volume 256, generated hydrogen gas can be transported into the interior volumes 258 of the membranes 252, where the sweep gas flow 218 and/or applied vacuum carries the hydrogen gas out of the reactor 250, for example, in a manner similar to that describe above for FIGS. 2A-2D and 2G-2H. In some embodiments, the sweep gas flow 218 through each interior volume 248 and/or vacuum pressure applied to each interior volume 248 can be substantially identical (e.g., identical sweep gas composition and/or flow rate). Alternatively, in some embodiments, the sweep gas flow 218 through one of the interior volumes 248 and/or vacuum pressure applied to one of the interior volumes 248 may be different than one or more of the other interior volumes.

Although a particular number and geometric arrangement of hollow fiber membranes 252 is shown in FIGS. 2E-2F, other numbers and/or geometric arrangements are also possible according to one or more contemplated embodiments. In some embodiments, the hollow fiber membranes can be disposed in an arrangement with increased packing density, for example, hexagonal close-packed (e.g., with a predetermined spacing maintained between adjacent fiber membranes to allow provision of catalyst). In the illustrated example, of FIGS. 2E-2F, the outer volume 256 is provided with the catalyst 210 and is used as the reaction volume, and the interior volumes 258 are used as the sweep volume. However, in some embodiments, the reaction can instead occur within the interior volume of each hollow fiber membrane, for example, in a manner similar to that described above with respect to FIGS. 2C-2D. Alternatively, in some embodiments, the catalyst can be disposed within a porous region of the hollow fiber membrane, and the reaction can occur within the porous region, in a manner similar to that described above with respect to FIGS. 2G-2H.

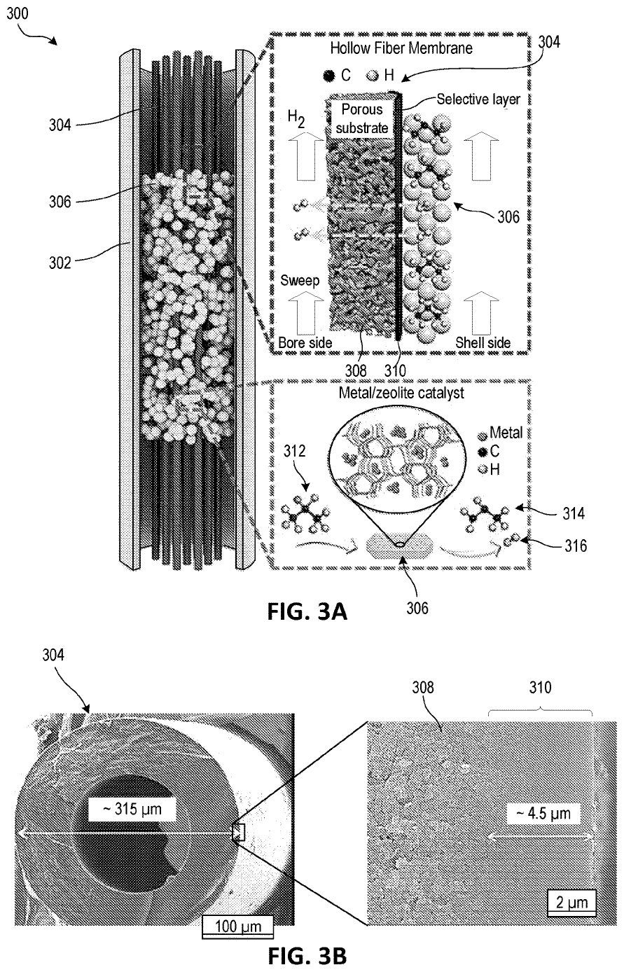

FIG. 3A illustrates another catalytic membrane reactor 300 having a plurality of hollow fiber membranes 304 and metal/zeolite catalyst 306 within a reactor housing 302. Each hollow fiber membrane 304 can have an asymmetric structure comprising a porous substrate layer 308 disposed adjacent to the bore side and a dense selective layer 310 disposed adjacent to the shell side. For example, the dense selective layer 310 can have a thickness less than (e.g., no more than 10% of) a thickness of the porous substrate layer 308, as shown in FIG. 3B. The metal/zeolite catalyst 306 can be disposed within the reactant flow volume on the shell side, for example, adjacent to the dense selective layer 310.

In some embodiments, the catalytic membrane reactor 300 can be used for alkane dehydrogenation. For example, a reactant flow comprising an alkane 312 can be provided on the shell side. The alkane 312 can interact with the catalyst 306 at an elevated temperature (e.g., 300-1000° C.), thereby converting the alkane 312 into hydrogen gas 316 and an alkene 314. The generated hydrogen gas 316 can then be transported through layers 310, 308 of the hollow fiber membrane 304 into the bore side volume, where the transported hydrogen is carried away by the sweep gas flow in the bore side.

### Catalytic Membrane Reactor Systems

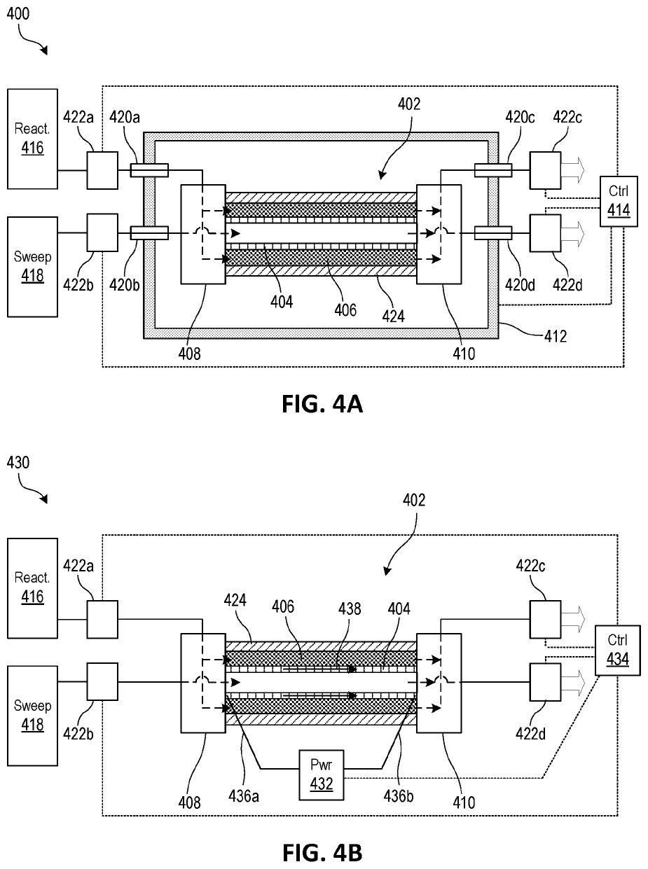

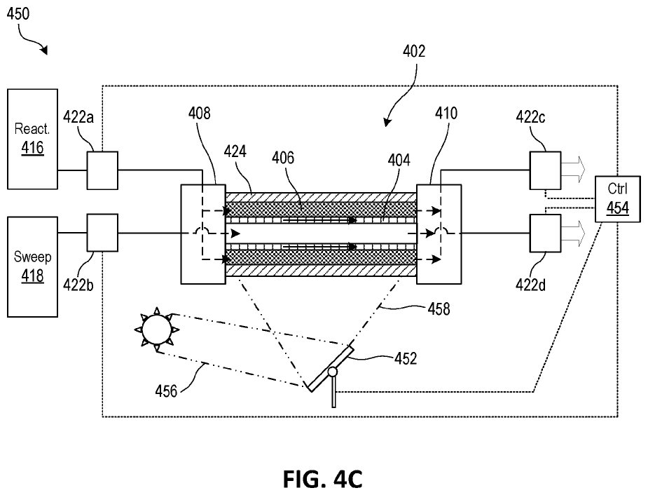

FIG. 4A shows an exemplary system 400 employing a catalytic membrane reactor 402 for conversion of one or more reactants. The catalytic membrane reactor 402 can include first and second gas volumes separated by an annular wall 404 and one or more catalysts 406 within a reactor housing 424, for example, as described above with respect to any of FIGS. 1A-2H.
The catalytic membrane reactor 402 can be disposed within a furnace 412. In some embodiments, the furnace 412 can heat the catalytic membrane reactor to a reaction temperature within a range of 100-1000° C., inclusive (e.g., ≤600° C. and/or ≥300° C.), for example, via combustion of gasoline, natural gas, coal, biogas, and/or biomass.

The one or more reactants (e.g., alkane or other hydrocarbon) can be provided from one or more feed gas sources 416 to the catalytic membrane reactor 402 via an inlet manifold 408 and/or a respective inlet line that passes through a wall of the furnace 412, for example, via respective heat seal 420a. The resulting products and/or any remaining unconverted reactants can be removed from the first volume via outlet manifold 410 and/or a respective outlet line that passes through a wall of the furnace 412, for example, via respective heat seal 420c. The products can be separated from the unreacted reactants for subsequent use or storage, while the unconverted reactants can be recirculated back to source 416 and/or the first gas volume of the reactor 402 for conversion.

At a same time as the feed gas flow in the first gas volume of the catalytic membrane reactor 402, a sweep gas flow can be provided in the second gas volume of the reactor 402. The sweep gas can be provided from one or more sweep gas sources 418 to the reactor 402 via the inlet manifold 408 (or a separate manifold) and/or a respective inlet line that passes through a wall of the furnace 412, for example, via respective heat seal 420b. For example, the sweep gas can comprise an inert gas that does not react with the hydrogen gas transported from the first volume to the second volume. The sweep gas and transported H₂ can be removed from the second gas volume of the catalytic membrane reactor 402 via outlet manifold 410 (or a separate manifold) and/or a respective outline line that passes through a wall of the furnace 412, e.g., via respective heat seal 420d. The H₂ can be separated from the sweep gas for subsequent use or storage, while the sweep gas can be recirculated back to source 418 and/or the second gas volume of the catalytic membrane reactor 402 for repeated use.

In the illustrated example of FIG. 4A, the system 400 includes a control system 414 that can regulate operation of the system 400. In some embodiments, each of the input and output lines can include a respective gas flow control/sensing module 422a-422d, which may include, for example, valves, temperature sensors, temperature controllers, pumps, and/or other devices to monitor and/or control the variables of gas flow rates, reaction temperatures, and/or feed and sweep gas compositions to optimize or otherwise control reactant conversion and/or product formation. In some embodiments, the control system 414 can modify flow rates, feed gas composition, sweep gas composition, and/or temperature to regulate reactant conversion efficiency and/or product selectivity, for example, by controlling the gas flow control/sensing modules 422a-422d and/or by controlling furnace 412.

In some embodiments, the catalytic membrane reactor can be heated to the reaction temperature without use of, or without solely relying on, conventional fossil-fuel-based furnace heating. Rather, in some embodiments, a furnace in which the catalytic membrane reactor is disposed and/or a portion of the catalytic membrane reactor can be heated by electricity-based heating (e.g., via Joule heating). For example, in some embodiments, the electro-conductive nature of the CMS membrane can allow it to operate as a Joule heating element, such as by passing an electrical current through the CMS membrane. In some embodiments, the electrical current can be generated using renewable techniques (e.g., wind, photovoltaic, solar thermal, hydropower, geothermal, etc.).

For example, FIG. 4B shows a system 430 employing Joule heating to provide the reaction temperature. Similar to system 400 in FIG. 4A, system 430 can include a catalytic membrane reactor 402, an inlet manifold 408, an outlet manifold 410, one or more feed gas sources 416, one or more sweep gas sources 418, gas flow control/sensing modules 422a-422d, and reactor housing 424. However, instead of using a furnace to heat the catalytic membrane reactor 402, system 430 can include an electrical power source 432 (e.g., DC power supply) connected to respective portions of annular wall 404 (e.g., axially separated surface portions) via respective electrical connections 436a, 436b (e.g., leads). Alternatively or additionally, in some embodiments, electrical connection between the electrical power source 432 and the annular wall 404 can be made via manifolds 408, 410.

Application of a voltage across the annular wall 404 via electrical connections 436a, 436b can cause an electrical current 438 to pass therethrough (e.g., substantially parallel to an axial direction of the annular wall), which electrical current 438 causes Joule heating of the annular wall 404. In the illustrated example of FIG. 4B, the system 430 includes a control system 434 that can regulate operation of the system 430. In some embodiments, the control system 434 can modify flow rates, feed gas composition, sweep gas composition, and/or temperature to regulate reactant conversion efficiency and/or product selectivity, for example, by controlling the gas flow control/sensing modules 422a-422d and/or by controlling electrical power source 432. For example, the electrical power source 432 can be controlled to Joule heat the annular wall 404 to provide a reaction temperature within the first volume of the catalytic membrane reactor 402 in a range of 100-1000° C. (e.g., ≤600° C. and/or ≥300° C.).

In the illustrated example of FIG. 4B, the electrical current 438 is passed through the annular wall 404 to effect Joule heating thereof. Alternatively or additionally, in some embodiments, other components of the catalytic membrane reactor 402 can be subjected to Joule heating to provide the reaction temperature. For example, the electrical power source 432 (or a different power source) can be connected to portions of reactor housing 424, and application of a voltage thereto can cause an electrical current to pass through the reactor housing 424, which electrical current causes Joule heating of the reactor housing (e.g., to provide a reaction temperature within the first volume of the catalytic membrane reactor 402 in a range of 100-1000° C.).

Alternatively or additionally, in some embodiments, the heating can be provided by solar radiation. For example, in some embodiments, the catalytic membrane reactor can be heated by direct or indirect solar thermal heating. With direct solar thermal heating, solar radiation can be focused and/or concentrated on the catalytic membrane reactor or a furnace in which the reactor is disposed. With indirect solar thermal heating, solar radiation can be focused and/or concentrated on a receiver to heat a heat-transfer fluid (e.g., water or molten salt) flowing therein, and the heated heat-transfer fluid is then used to heat the catalytic membrane reactor or a furnace in which the reactor is disposed.

For example, FIG. 4C shows a system 450 employing solar thermal heating to provide the reaction temperature. Similar to system 400 in FIG. 4A, system 450 can include a catalytic membrane reactor 402, an inlet manifold 408, an outlet manifold 410, one or more feed gas sources 416, one or more sweep gas sources 418, gas flow control/sensing modules 422a-422d, and reactor housing 424. However, instead of using a furnace to heat the catalytic membrane reactor 402, system 450 can include one or more concentrating devices 452 (e.g., heliostats, lenses, etc.) that redirect and/or focus insolation 456 to illuminate the catalytic membrane reactor 402. For example, the solar radiation 458 can be directed (e.g., reflected) by the concentrating device(s) 452 onto one or more exposed surfaces of the catalytic membrane reactor 402, for example, the reactor housing 424. Alternatively or additionally, the reactor housing 424 can be formed of an optically transparent material, such that the directed solar radiation 458 passes through the housing 424 and is incident on the catalyst 406 and/or annular wall 404.

In the illustrated example of FIG. 4C, the system 450 includes a control system 454 that can regulate operation of the system 450. In some embodiments, the control system 454 can modify flow rates, feed gas composition, sweep gas composition, and/or temperature to regulate reactant conversion efficiency and/or product selectivity, for example, by controlling the gas flow control/sensing modules 422a-422d and/or by controlling concentrating device(s) 452. For example, the concentrating device(s) 452 can be controlled to heat the catalytic membrane reactor 402 to provide a reaction temperature within the first volume of the catalytic membrane reactor 402 in a range of 100-1000 ° C.(e.g., ≤600° C. and/or ≥300° C.).

Although FIGS. 4A-4C show certain heating configurations, embodiments of the disclosed subject matter are not limited thereto. Rather, other heating configurations are also possible according to one or more embodiments. Moreover, although the heating configurations are illustrated separately in FIGS. 4A-4C, the heating configurations can be combined in some embodiments. In some embodiments, the sweep gas flow (e.g., from sweep gas source 418) in any of FIGS. 4A-4C can be replaced or supplemented by vacuum application, for example, to the second gas volume of catalytic membrane reactor 402 (e.g., via outlet manifold 410).

Alternatively or additionally, in some embodiments, the reactant gas flow (e.g., from feed gas source 416) in any of FIGS. 4A-4C can be replaced or supplemented by vacuum application, for example, to the first gas volume of catalytic membrane reactor 402 (e.g., via outlet manifold 410).

### Catalytic Membrane Reactor Methods

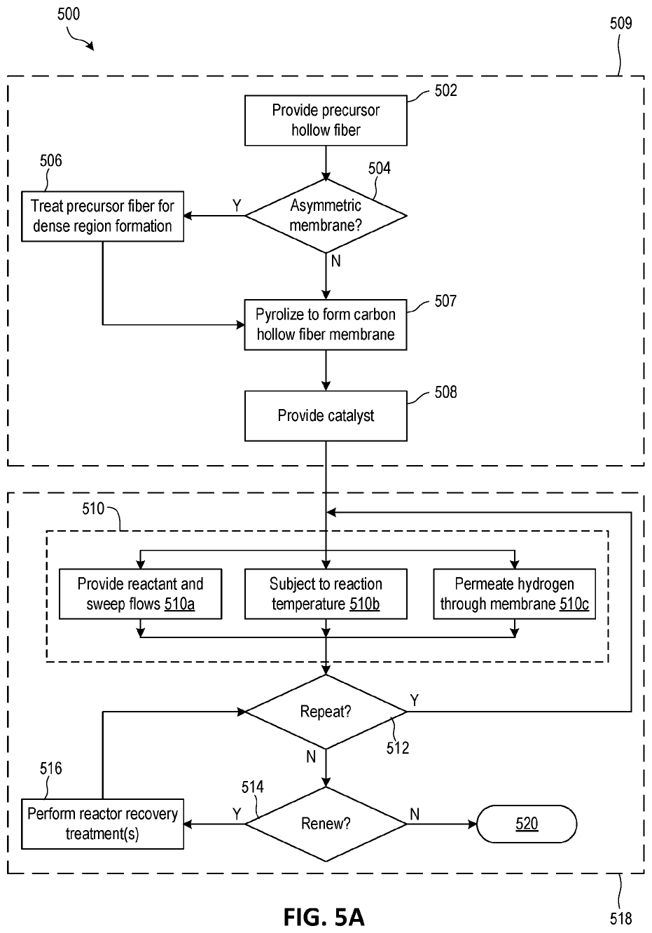

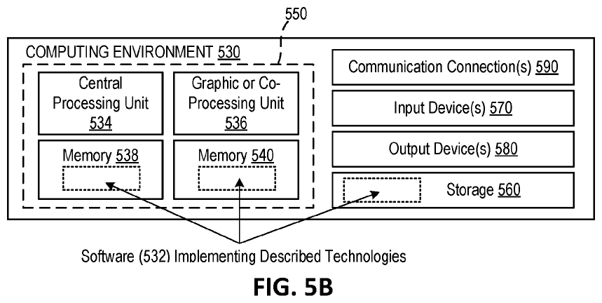

FIG. 5A illustrates a method 500 for fabrication and use of a catalytic membrane reactor. The method 500 can initiate at process block 502, where one or more precursor hollow fibers can be provided. In some embodiments, the precursor hollow fiber can comprise a mixed matrix material, for example, comprising metals, metal oxides, zeolites, alumina, silica, metal-organic frameworks, zeolitic imidazolate frameworks, covalent organic frameworks, graphene, graphene oxide, Mxenes, and/or carbon nanotubes. Alternatively or additionally, in some embodiments, the precursor hollow fiber can comprise one or more polymers, such as but not limited to polysulfones, polyethersulfones, polyetherketones, polyimides, polyetherimides, polyamides, polyamide-imides, polyesters, polybenzimidazoles, polybenzobenzimidazoles, polyethers, or any combination thereof. In some embodiments, the provision of process block 502 can include forming the one or more precursor hollow fibers. For example, the precursor hollow fibers can be formed by a dry-jet/wet-quench fiber spinning process.

The method 500 can proceed to decision block 504. If an asymmetric hollow fiber membrane is desired at decision block 504, the method 500 can proceed to process block 506, where the one or more precursor hollow fibers are subjected to a pretreatment for dense region formation. In some embodiments, the pretreatment of process block 506 can include exposing the precursor hollow fiber to a silane, such as vinyltrimethoxysilane (VTMS). For example, precursor hollow fibers can be soaked in a 10 wt % VTMS/hexane solution for 24 hours and then exposed to water-vapor saturated air for another 24 hours.

After the pretreatment of process block 506, or if an asymmetric hollow fiber membrane is not desired at decision block 504, the method 500 can proceed to process block 507, where the one or more precursor hollow fibers are pyrolyzed to convert the one or more precursor fibers into one or more hollow fiber membranes. For example, the pyrolyzing of process block 507 can be at a temperature of at least 500° C. In some embodiments, process block 507 can include selecting a temperature for pyrolysis to provide a pore size of the annular wall of the hollow fiber membrane for desired product selectivity. For example, higher pyrolysis temperatures (e.g., ~675° C.) can result in smaller pore sizes while lower pyrolysis temperatures (e.g., ~550° C.) can result in larger pore sizes. In some embodiments, after the pyrolyzing of process block 507, the one or more hollow fiber membranes can be formed of carbon (e.g., consisting essentially of carbon). If the one or more precursor hollow fibers were pretreated via process block 506, the one or more hollow fiber membranes resulting from the pyrolyzing of process block 507 can have an asymmetric configuration, for example, with a more porous first region (e.g., radially inner annular region) and a denser second region (e.g., radially outer annular region).

At process block 508, one or more catalysts can be formed, loaded, or otherwise provided with respect to the one or more hollow fiber membranes (e.g., within or surrounding). In some embodiments, the one or more catalysts can include a dehydrogenation catalyst. For example, the dehydrogenation catalyst can comprise single atoms of metal, sub-nanometer-size clusters of metal, nanometer-size clusters of metal, or any combination thereof confined in a siliceous zeolite. In some embodiments, the metal of the dehydrogenation catalyst can be selected from the group consisting of Pt, Zn, Sn, Cu, Co, Pd, Fe, Ni, Ru, Rh, Ir, Mn, Y, Bi, Zr, Cr, Cs, Li, Na, K, Ca, Ba, and combinations thereof. In some embodiments, a concentration of metal in the dehydrogenation catalyst can be in a range of 0.001 wt % to 30 wt %, inclusive. In some embodiments, the dehydrogenation catalyst is selected from the group consisting of MFI zeolite, MWW zeolite, FER zeolite, FAU zeolite, BEA zeolite, MOR zeolite, and combinations thereof.

In some embodiments, the provision of process block 508 can include disposing one or more catalysts within the interstitial space between a reactor housing and the one or more hollow fiber membranes. Alternatively, in some embodiments, the provision of process block 508 can include disposing one or more catalysts within the interior volumes of the one or more hollow fiber membranes. Alternatively, in some embodiments, the provision of process block 508 can include disposing one or more catalysts within porous regions (e.g., an annular porous support layer) of the one or more hollow fiber membranes. In some embodiments, the provision of process block 508 can include forming the one or more catalysts. For example, the one or more catalysts can be formed by hydrothermal synthesis in an aqueous, sol-gel, or dry state, by dealumination followed by metal precursor impregnation, by in-situ reduction, or any combination of the foregoing.

At process block 510, the assembled catalytic membrane reactor (comprising the one or more hollow fiber membranes and the one or more catalysts) can be used to convert one or more reactants to one or more products, for example, via a non-oxidative dehydrogenation reaction.

For example, process block 510 can include sub-process blocks 510a-510c performed substantially simultaneously (e.g., concurrently). At sub-process block 510a, a flow of one or more reactants can be provided in a first gas volume of the catalytic membrane reactor, for example, via positive pressure applied to a first inlet of the catalytic membrane reactor and/or a vacuum pressure applied to a first outlet of the catalytic membrane reactor. In some embodiments, sub-process block 510a can include providing a flow of a sweep gas in a second gas volume of the catalytic membrane reactor, for example, via positive pressure applied to a second inlet of the catalytic membrane reactor and/or a vacuum pressure applied to a second outlet of the catalytic membrane reactor. Alternatively, in some embodiments, sub-process block 510a can include evacuating gas from the second gas volume of the catalytic membrane reactor, for example, via a vacuum pressure applied to the second outlet of the catalytic membrane reactor.

At sub-process block 510b, the catalytic membrane reactor can be subjected to a reaction temperature (e.g., an elevated temperature). For example, the reaction temperature can be in a range of 100-1000° C. (e.g., ≤600° C. and/or ≥300° C.). In some embodiments, the reaction temperature can be generated via furnace heating (e.g., via combustion of gasoline, natural gas, coal, biogas, and/or biomass), solar heating (e.g., via focused solar radiation), and/or electrical heating (e.g., via Joule heating of one or more components of the catalytic membrane reactor). In some embodiments, interaction of the one or more reactants with the one or more catalysts at the reaction temperature can convert at least some of the reactants (e.g., a hydrocarbon) into hydrogen gas and one or more additional products (e.g., a corresponding olefin). At sub-process block 510c, the produced hydrogen gas can be transported from the first gas volume, through the annular wall of the hollow fiber membrane of the catalytic membrane reactor, into the second gas volume for removal from the catalytic membrane reactor. In some embodiments, the transport of process block 510c can be via bulk diffusion, i.e., ion transport without application of an external electric field.

The method 500 can proceed from process block 510 to decision block 512, where it is determined if the conversion reaction should be repeated. If repetition is desired, the method 500 can return to process block 510. In some embodiments, the repeating of the conversion reaction can comprise multiple passes by separating out unreacted reactants from an output flow of the catalytic membrane reactor (e.g., a product flow) and returning the unreacted reactants to an inlet of the catalytic membrane reactor (e.g., a reactant flow). Alternatively or additionally, in some embodiments, the repeating of the conversion reaction can comprise repeating process block 510 using new or additional reactants in the reactant flow. Alternatively or additionally, the repeating of the conversion reaction can reflect a substantially continuous operation of the catalytic membrane reactor, for example, for at least tens (10s) or hundreds (100s) of hours.

Otherwise, the method 500 can proceed from decision block 512 to decision block 514, where it is if the catalytic membrane reactor should be renewed. If renewal is desired, the method 500 can proceed to process block 516, where a reactor recovery treatment can be performed, for example, to regenerate or recover a catalyst in the catalytic membrane reactor.

For example, the recovery treatment of process block 516 can include exposing the catalyst to H₂ gas flow at about 500° C. for about 1 hour. Alternatively or additionally, the recovery treatment of process block 516 can include subjecting the catalyst to a calcination treatment in air. After the recovery treatment, the method 500 can return to decision block 512. If renewal is not desired at decision block 514, the method 500 can optionally proceed to terminal block 520, where the method can terminate.

Although blocks 502-520 of method 500 have been described as being performed once, in some embodiments, multiple repetitions of a particular process block may be employed before proceeding to the next decision block or process block. In addition, although blocks 502-520 of method 500 have been separately illustrated and described, in some embodiments, process blocks may be combined and performed together (simultaneously or sequentially).

Indeed, in some embodiments, sub-process blocks 510a-510c may be performed simultaneously and continuously (e.g., for hundreds or thousands of hours), for example, to convert a stream of reactants into products. Moreover, although FIG. 5A illustrates a particular order for blocks 502-520, embodiments of the disclosed subject matter are not limited thereto. Indeed, in certain embodiments, the blocks may occur in a different order than illustrated or simultaneously with other blocks.

In some embodiments, method 500 can include steps or other aspects not specifically illustrated in FIG. 5A. Alternatively or additionally, in some embodiments, method 500 may comprise only some of blocks 502-520 of FIG. 5A. For example, in some embodiments, method 500 can include some or all of blocks 502-508 (e.g., with or without catalyst provision 508), e.g., to perform a fabrication method 509 for one or more catalytic membrane reactors. Alternatively or additionally, in some embodiments, method 500 can include some or all of blocks 510-520 (e.g., with or without the reactor recovery of blocks 514-516), e.g., to perform a reaction method 518 that converts one or more reactants into one or more products.

### Computer Implementation

FIG. 5B depicts a generalized example of a suitable computing environment 530 in which the described innovations may be implemented, such as but not limited to aspects of control system 414, power source 432, control system 434, control system 454, and/or method 500. The computing environment 530 is not intended to suggest any limitation as to scope of use or functionality, as the innovations may be implemented in diverse general-purpose or special-purpose computing systems. For example, the computing environment 530 can be any of a variety of computing devices (e.g., desktop computer, laptop computer, server computer, tablet computer, etc.).

With reference to FIG. 5B, the computing environment 530 includes one or more processing units 534, 536 and memory 538, 540. In FIG. 5B, this basic configuration 550 is included within a dashed line. The processing units 534, 536 execute computer-executable instructions. A processing unit can be a central processing unit (CPU), processor in an application-specific integrated circuit (ASIC), or any other type of processor (e.g., hardware processors, graphics processing units (GPUs), virtual processors, etc.). In a multi-processing system, multiple processing units execute computer-executable instructions to increase processing power. For example, FIG. 5B shows a central processing unit 534 as well as a graphics processing unit or co-processing unit 536. The tangible memory 538, 540 may be volatile memory (e.g., registers, cache, RAM), non-volatile memory (e.g., ROM, EEPROM, flash memory, etc.), or some combination of the two, accessible by the processing unit(s). The memory 538, 540 stores software 532 implementing one or more innovations described herein, in the form of computer-executable instructions suitable for execution by the processing unit(s).

A computing system may have additional features. For example, the computing environment 530 includes storage 560, one or more input devices 570, one or more output devices 580, and one or more communication connections 590. An interconnection mechanism (not shown) such as a bus, controller, or network interconnects the components of the computing environment 530. Typically, operating system software (not shown) provides an operating environment for other software executing in the computing environment 530, and coordinates activities of the components of the computing environment 530.

The tangible storage 560 may be removable or non-removable, and includes magnetic disks, magnetic tapes or cassettes, CD-ROMs, DVDs, or any other medium which can be used to store information in a non-transitory way, and which can be accessed within the computing environment 530. The storage 560 can store instructions for the software 532 implementing one or more innovations described herein.

The input device(s) 570 may be a touch input device such as a keyboard, mouse, pen, or trackball, a voice input device, a scanning device, or another device that provides input to the computing environment 530. The output device(s) 380 may be a display, printer, speaker, CD-writer, or another device that provides output from computing environment 530.

The communication connection(s) 590 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, radio-frequency (RF), or another carrier.

Any of the disclosed methods can be implemented as computer-executable instructions stored on one or more computer-readable storage media (e.g., one or more optical media discs, volatile memory components (such as DRAM or SRAM), or non-volatile memory components (such as flash memory or hard drives)) and executed on a computer (e.g., any commercially available computer, including smart phones or other mobile devices that include computing hardware). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or any other such network) using one or more network computers.

For clarity, only certain selected aspects of the software-based implementations are described. Other details that are well known in the art are omitted. For example, it should be understood that the disclosed technology is not limited to any specific computer language or program. For instance, aspects of the disclosed technology can be implemented by software written in C++, Java™, Python®, and/or any other suitable computer language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means. In any of the above-described examples and embodiments, provision of a request (e.g., data request), indication (e.g., data signal), instruction (e.g., control signal), or any other communication between systems, components, devices, etc. can be by generation and transmission of an appropriate electrical signal by wired or wireless connections.

### Fabricated Examples and Experimental Results

### Catalyst and Membrane Design

Platinum-zinc/silicalite-1 (Pt—Zn/S1) catalyst was prepared by in-situ hydrothermal synthesis. Pt precursor (i.e., [Pt(NH₂CH₂CH₂NH₂)₂]Cl₂) solution was prepared by dissolving 0.24 g of PtCl₂ in an aqueous solution containing 9.00 g of deionized (DI) water and 1.15 g of ethylenediamine. Zn precursor (i.e., [Zn(NH₂CH₂CH₂NH₂)₃](acac)₂) solution was prepared by dissolving 0.84 g of Zn(acac)₂·xH₂O in an aqueous solution containing 8.00 g of DI water and 2.32 g of ethylenediamine. 2.64 g of [Pt(NH₂CH₂CH₂NH₂)₂]Cl₂ solution was added into a TPAOH solution that was prepared by mixing 26.00 g of 40 wt % TPAOH with 30.00 g of DI water. The mixture was stirred for 10 minutes followed by addition of 16.64 g TEOS. After continuous stirring for 6 hours, 2.98 g of [Zn(NH₂CH₂CH₂NH₂)₃](acac)₂ solution was added.

The mixture was further stirred for 30 minutes and then transferred into Teflon lined stainless steel autoclaves. The autoclaves were placed in a convection oven at 170° C. for 3 days under static conditions. After the hydrothermal synthesis, the sample was collected by centrifugation and washed by DI water. The centrifugation and DI water washing steps were repeated until pH of the washing solution was about 9. The sample was dried in a convection oven at 70° C. overnight. The dried catalyst sample was pressed into a pellet, crushed, and sieved to provide a particle size in a range of about 180-425 μm, inclusive.

Matrimid® precursor hollow fibers were formed using a dry-jet/wet-quench fiber spinning process. The dope composition of the Matrimid® precursor hollow fiber was 26.2 w t% Matrimid® 5218 polyimide powder (dried under vacuum at 110° C. overnight before dope preparation), 53.0 wt % n-methyl-pyrrolidone (NMP), 5.9 wt % tetrahydrofuran (THF), and 14.9 wt % ethanol. The fibers were formed using a spinning temperature of 60° C., a quench bath temperature of 50° C., a dope fluid flow rate of 180 mL/hour, a bore fluid flow rate of 60 mL/hour, a bore fluid composition of 90 wt % NMP and 10 wt % DI water, an air gap height of 10 cm, and a fiber take-up rate of 20 m/minute. The as-spun polymer hollow fibers were sequentially soaked in three (3) separate DI water baths over the course of 72 hours, three (3) separate methanol baths for 20 minutes each, and three (3) separate hexane baths for 20 minutes each. The polymer hollow fibers were dried in a fume hood overnight before being dried under vacuum at 75° C. for 12 hours.

Silane pretreatment of precursor hollow fibers was performed to resist substrate collapse of CMS hollow fiber membranes during pyrolysis. The precursor hollow fibers were soaked in a 10 wt % vinyltrimethoxysilane (VTMS)/hexane solution for 24 hours and then exposed to water-vapor saturated air for another 24 hours. The VTMS-treated precursor hollow fibers were vacuum dried at 150° C. for 12 hours prior to pyrolysis. The treated precursor hollow fibers were placed into a U-shaped quartz reactor (outer diameter (OD): 6.35 mm; inner diameter (ID): 4.35 mm), and the quartz-reactor was then loaded into a straight quartz tube (OD: 60 mm; ID: 54 mm). The quartz tube, with U-shaped reactor therein, was then heated in a three-zone tube furnace. Ultra-high purity argon (Ar) was introduced to the quartz tube at 500 mL/minute via a mass flow controller. The system oxygen level was kept below 5 ppm prior to pyrolysis, which was monitored by an oxygen analyzer.

In the pyrolysis process, the precursor fibers were first heated from room temperature to 250° C. at a rate of 0.22° C./second. The temperature was then increased to T_{final}-15 (where T_{final} is the final pyrolysis temperature) at a rate of 0.0642° C./second, followed by increasing to T_{final} at a rate of 0.00417° C./second. The value of T_{final} was set as 550° C. for CMS hollow fiber membranes for propane dehydrogenation and 675° C. for CMS membranes for ethane dehydrogenation. The heating was held at T_{final} for 2 hours and then allowed to naturally cool to ambient (e.g., room temperature).

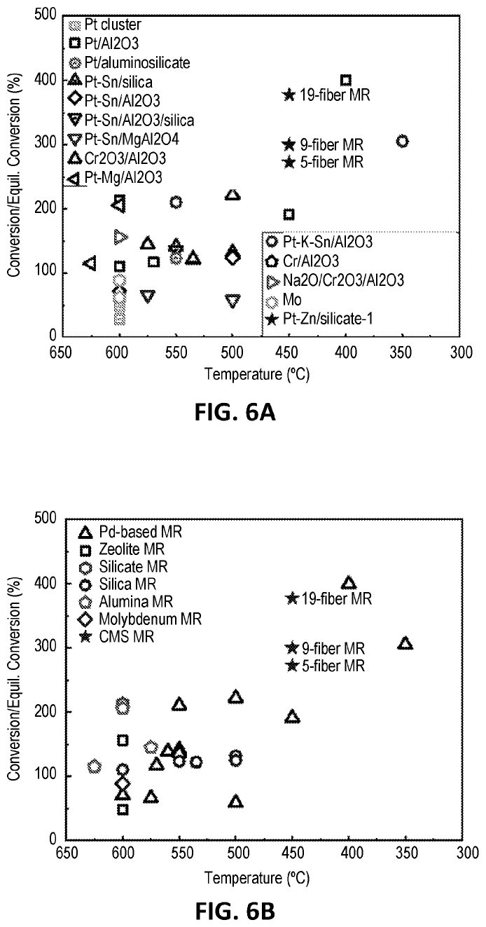

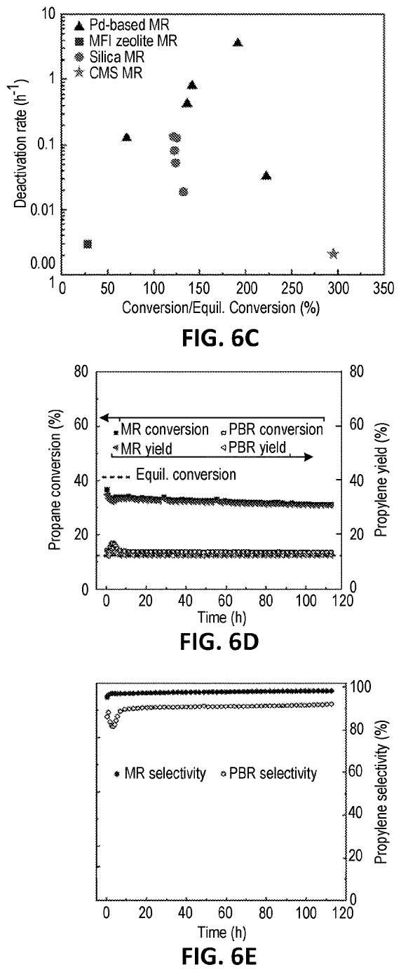

In comparison to catalysts and membranes used in conventional PDH MRs, the CMS MR reached up to 3 times as high propane equilibrium conversion, record-low deactivation rate, and excellent long-term durability, as shown by FIGS. 6A-6E.

### Propane Dehydrogenation

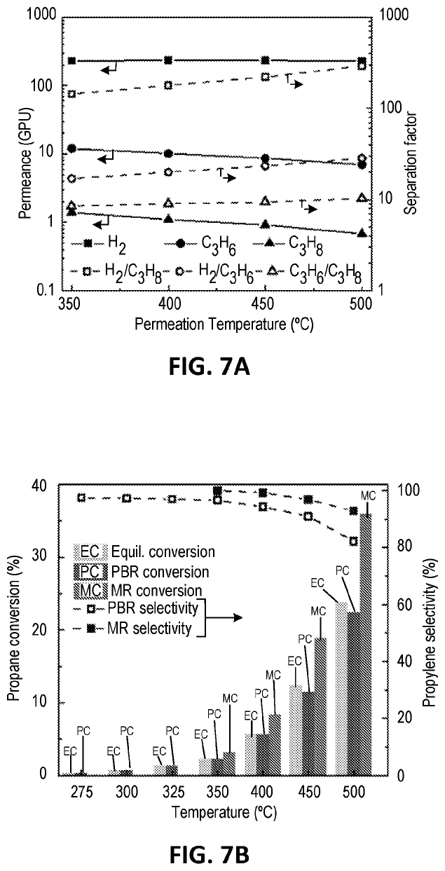

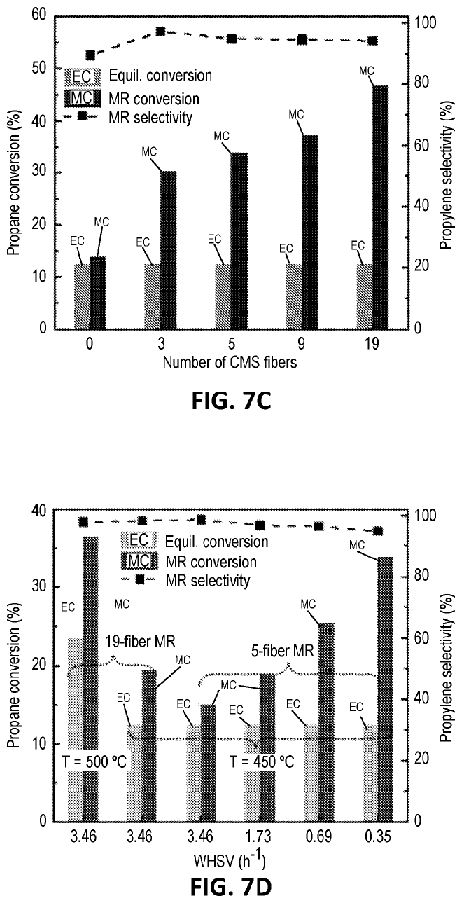

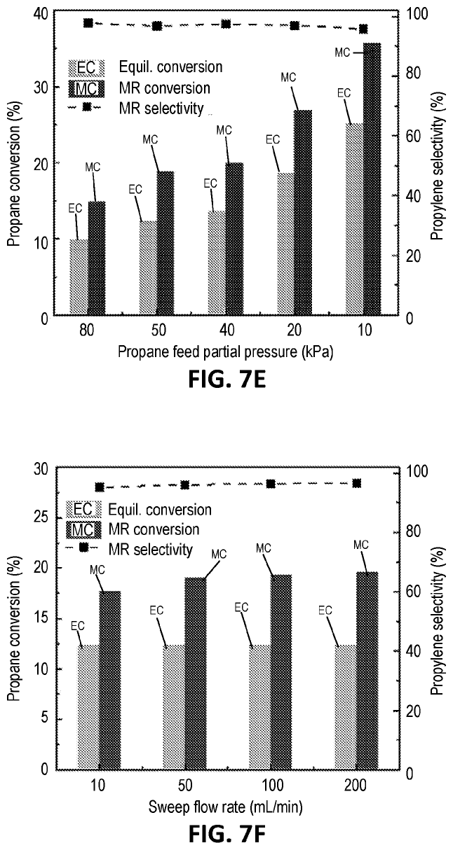

Hollow fiber membranes were made by pyrolysis (550° C.) of Matrimid® polyimide precursor hollow fiber membranes fabricated by dry-jet/wet-quench spinning. Precursor hollow fibers were pre-treated by VTMS to provide the asymmetric structure characterized by a porous substrate and a thin dense separation layer (~4.5 μm), as shown in FIG. 4B. This asymmetric structure gives the resulting hollow fiber membrane attractive H₂ permeance and high H₂/C₃H₈ separation factor that increases with operation temperature, as shown in FIG. 7A. Prior to the reaction test, the membrane reactor system was flushed with an Ar-gas flow (20 mL/minute) overnight. The Ar-gas flow on the shell side of the hollow fiber membrane was then switched to a mixture of H₂/N₂ (20 mL/minute; H₂: N₂=1:5; ultrapure, Air gas) to pretreat the catalyst at 500° C. (heat rate of 0.0167° C./second) for 3 hours prior to performing the reaction. The H₂/N₂-gas flow on the shell side was then switched back to the Ar-gas flow to flush out of residue H₂ gas in the reactor system. Afterward, the Ar-gas flow was switched to the alkane (CₓH₂ₓ₊₂, where x=3 for propane (C₃H₈) mixed with the Ar internal standard (ultra-high purity) to start the dehydrogenation reaction. The Ar-gas flow on the bore side of the membrane reactor was maintained during the catalyst pretreatment and catalysis testing steps.

The silicalite-1 supported platinum (Pt) and zinc (Zn) (Pt—Zn/S1) was used for the catalyst in the nonoxidative PDH reaction, which catalyst was hydrothermally synthesized.

Physiochemical property characterizations confirmed the structural integrity of the synthesized catalyst. The catalyst was tested in a packed-bed reactor (PBR). These tests confirmed that the catalyst had excellent stability (as shown in FIGS. 6D-6E) and low threshold activation temperature (as shown in FIG. 7C), as the PDH over the Pt—Zn/S1 catalyst attained equilibrium conversions at temperatures as low as 275° C. The CMS hollow fiber MR was assembled by packing the Pt—Zn/S1 catalyst on the shell side of CMS hollow fiber membranes (e.g., as shown in FIG. 4A). The PDH reaction was carried out by flowing a C₃H₈/Ar mixture on the shell side and an inert sweep (Ar) to carry away the H₂ permeate on the bore side.

FIGS. 7A-7F show the results of testing to study the roles of variables on C₃H₈ conversion and C₃H₆ selectivity. As shown in FIG. 7B, as the reaction temperature increased from 350 to 500° C., the C₃H₈ conversion increased from 3.2 to 36.0%, which can be explained by the endothermic nature of the reaction. Increasing the reaction temperature caused the C₃H₆ selectivity to drop from nearly 100.0% to 92.8%, possibly due to side reactions such as cracking and cyclization that can occur at higher temperatures. As the number of CMS hollow fibers was increased from 0 (i.e., PBR) to 19, the C₃H₈ conversion increased from 12.4% to 46.8%, as shown in FIG. 7C, which represented an almost quadrupling of the thermodynamic equilibrium conversion. The larger number of CMS hollow fibers can increase the membrane permeation area and hence the H₂ permeation flow rate. The higher H₂ permeation flow rate can reduce H₂ partial pressure in the reaction side, which can drive the reversible PDH reaction forward according to the Le Chatelier's principle. As the weight hourly space velocity (WHSV) of the propane feed decreased from 3.46 to 0.35 h⁻¹ at 450° C., C₃H₈ conversion increased and the C₃H₆ selectivity dropped. Membrane reactors with more CMS fibers (e.g., 19 fiber) and operated at higher temperature (e.g., 500° C.) can be used to achieve commercially attractive conversion (e.g., 36.5%) under high WHSV (e.g., 3.46 h⁻¹), as shown in FIG. 7D. As the C₃H₈ feed partial pressure decreased from 80 to 10 kPa, C₃H₈ conversion increased with almost constant C₃H₆ selectivity, as shown in FIG. 7E. As the sweep flow rate increased from 10 to 200 mL/minute, both C₃H₈ conversion and C₃H₆ selectivity increased, as shown in FIG. 7F. The enhanced conversion at higher sweep flow rates can be attributed to increased H₂ permeation flux, which was due to lower permeate H₂ partial pressure and increased H₂ permeation driving force. The higher H₂ permeation flux also reduces H₂ partial pressure in the reaction side, which suppresses side reactions such as hydrogenolysis and hence increases the C₃H₆ selectivity.

The above-noted parametric studies guided the optimal CMS MR operation conditions in the long-term PDH test, as shown in FIGS. 6D-6E. At 450° C., after time-on-stream (TOS) of ~5 hours (i.e., induction period), the MR achieved 34.1% C₃H₈ conversion, which was 2.4 times as high as that in PBR (14.0%). The MR also showed higher C₃H₆ selectivity (~97.1%) than the PBR (~89.4%). The enhanced conversion and selectivity allowed the CMS MR to improve C₃H₆ yield (i.e., 2.7 times as high as the PBR). It should be noted that the CMS MR had excellent stability for at least 110 hours with a slight drop of C₃H₈ conversion (34.1% to 31.3%) and slightly increased C₃H₆ selectivity (97.1% to 98.3%). The slight decrease in C₃H₈ conversions could be due to catalyst coking, which was observed in thermo-gravimetric analysis of spent Pt—Zn/S1 catalyst showing 1.9% weight loss. Analysis of the spent membrane reactor did not show obvious particle sintering in catalyst or morphology change in the CMS membrane.

The catalyst activity can be recovered by exposing the spent catalyst to an H₂ gas flow at 500° C. for 1 hour. Alternatively or additionally, a dual-zone fluidized bed membrane reactor design (e.g., a reactor based on the design of Medrano et al., “Two-zone fluidized bed reactor (TZFBR) with palladium membrane for catalytic propane dehydrogenation: Experimental performance assessment,” Industrial &Engineering Chemistry Research, 2013, 52: pp. 3723-31, which reactor design is hereby incorporated by reference herein) can be adapted for use in counteracting the catalyst deactivation/regeneration in continuous PDH operation, or for any other reason. The increase in C₃H₆ selectivity can be due to the suppression of cracking reactions by absence of catalyst acidity and lowering of H₂ pressure in the shell side. Remarkably, the activity of the PDH catalyst can be recovered by removal of carbon deposits via an H₂ gas stream. Compared with PDH MRs found in literature, the CMS MR excels by having high conversion enhancement (FIGS. 6A-6C), low operation temperature (FIG. 6B), and lowest deactivation rate (FIG. 6C).

### Ethane Dehydrogenation

Although the above described examples are directed to use of a CMS MR for PDH reactions, similar performance can be achieved for dehydrogenation of other alkanes, such as ethane. For ethane, the higher C—H bond stability (as compared to propane) can require a higher activation temperature for alkene production (i.e., C₂H₄). Moreover, the H₂/C₂H₆ gas pair (2.6 vs 3.9 Å) is more closely spaced than the H₂/C₃H₈ pair (2.6 vs 4.1 521 ). Accordingly, the CMS membrane and M/zeolite catalyst in the reactor were customized for ethane dehydrogenation EDH. In particular, hollow fiber membranes were made by pyrolysis (675° C.) of Matrimid® polyimide precursor hollow fiber membranes fabricated by dry-jet/wet-quench spinning. The higher temperature pyrolysis provided the membranes with smaller pores that can provide a more attractive separation factor for the H₂/C₂H₆ gas pair. In addition, cobalt in dealuminated beta zeolite (Co/deAl-BEA) was selected as the M/zeolite catalyst.

The precursor hollow fibers were pre-treated by VTMS to provide the asymmetric structure characterized by a porous substrate and a thin dense separation layer (~4.5 μm), as shown in FIG. 4B. Prior to the reaction test, the membrane reactor system was flushed with an Ar-gas flow (20 mL/minute) overnight. The Ar-gas flow on the shell side of the hollow fiber membrane was then switched to a mixture of H₂/N₂(20 mL/minute; H₂:N₂=1:5; ultrapure, Air gas) to pretreat the catalyst at 500° C. (heat rate of 0.0167° C./second) for 3 hours prior to performing the reaction. The H₂/N₂-gas flow on the shell side was then switched back to the Ar-gas flow to flush out of residue H₂ gas in the reactor system. Afterward, the Ar-gas flow was switched to the alkane (CₓH₂ₓ₊₂, where x=3 for propane (C₃H₈) mixed with the Ar internal standard (ultra-high purity) to start the dehydrogenation reaction. The Ar-gas flow on the bore side of the membrane reactor was maintained during the catalyst pretreatment and catalysis testing steps.

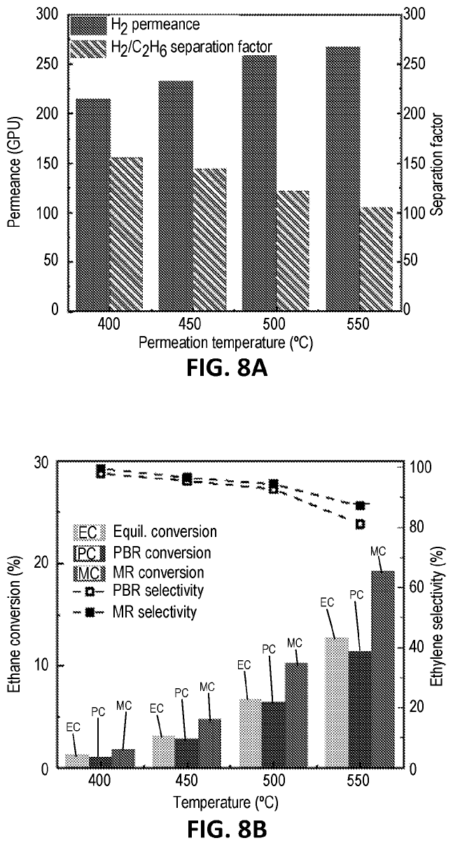

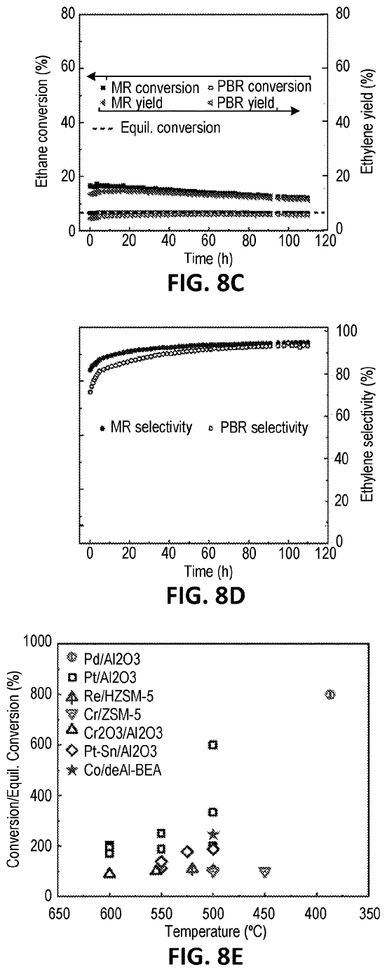

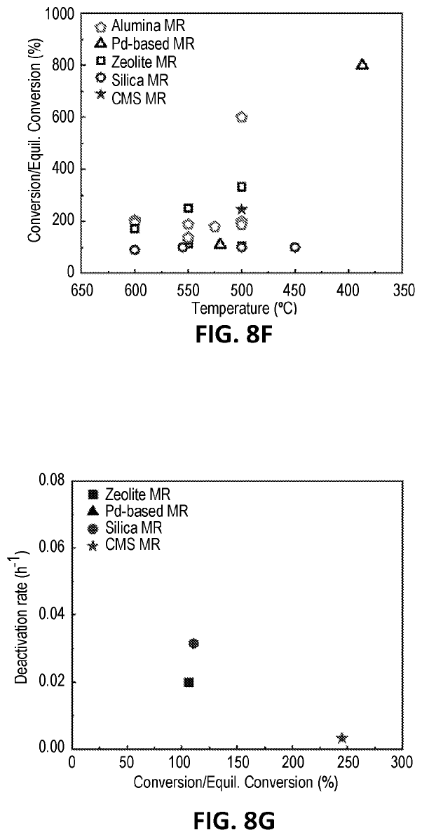

The Co/deAl-BEA catalyst attained near-equilibrium conversion in PBR at temperature as low as 400° C. (as shown in FIG. 8B) and showed no deactivation in a long-term stability test (as shown in FIGS. 8C-8D). At 500° C., in the initial stage of the time-on-stream (TOS) test, the CMS MR showed 16.6% C₂H₆ conversion, 2.5 times as high as the equilibrium conversion (6.7%). The C₂H₄ selectivity was also higher in the CMS MR (82.6%) than the PBR (71.3%), similar to the PDH study of FIGS. 6D-6E. A drop in C₂H₆ conversion (16.6% to 12.4%) accompanied with an increase in C₂H₄ selectivity (82.6% to 94.4%) was observed after the TOS of ~110 h, as shown in FIGS. 8C-8D. Compared to PDH, the conversion drop in EDH was more noticeable, which could be due to more carbonaceous deposit in the catalyst at the higher operation temperature. Notably, the EDH catalyst can fully recover its activity after calcination treatment in air. As shown in FIGS. 8E-8G, the CMS MR showed high C₂H₆ conversion enhancement, low operation temperature, and the lowest deactivation rate than EDH MRs reported in literature.

### Electrified Heating

To reduce CO₂ emissions from heating endothermic alkane dehydrogenation reactors, Joule heating of the CMS hollow fiber membrane was employed. The CMS hollow fiber membrane can be electrically conductive by virtue of its conjugated electronic structure. To electrify the MR, a CMS hollow fiber membrane was assembled with electrodes. The assembly was then placed in a quartz tube housing with a catalyst loaded on the shell side of the CMS hollow fiber membrane. To provide the Joule heating of the CMS hollow fiber, a design was employed that was different from that using process heat from an external furnace. A straight reactor module was used in the experiment. The CMS hollow fiber was prepared according to the procedure for PDH described above. The Pt—Zn/S1 catalyst was pretreated using the setup for PBR testing.

The CMS hollow fiber and PDH catalyst were then assembled into the straight quartz reactor (OD: 6.35 mm) using stainless steel Swagelok® fittings. Copper wire and the CMS hollow fiber were connected with two pieces of copper foil (one on each side). The copper foil served as a bridge to electrically connect the copper wire with CMS hollow fiber, where the copper wire did not go through the catalyst bed. The connected copper wire and CMS hollow fiber (length: 150 mm) were placed into the quartz tube (length: 200 mm), and the quartz tube connected to two Swagelok® Ultra-Torr Tee fittings (one at each side). Two short pieces of quartz tube were connected to the second opening of these two Ultra-Torr Tees. In order to acquire the reactor temperature, a thermocouple (K-type, OD: 0.25 mm) was placed into a silica capillary tube (ID: 0.53 mm) with one end closed. The silica capillary tube together with the thermocouple inside was aligned next to the CMS hollow fiber in the quartz reactor. Both ends of the CMS hollow fiber were connected to the silica capillaries for sweep gas delivery. Epoxy was applied to all the connection positions to avoid gas leakage.

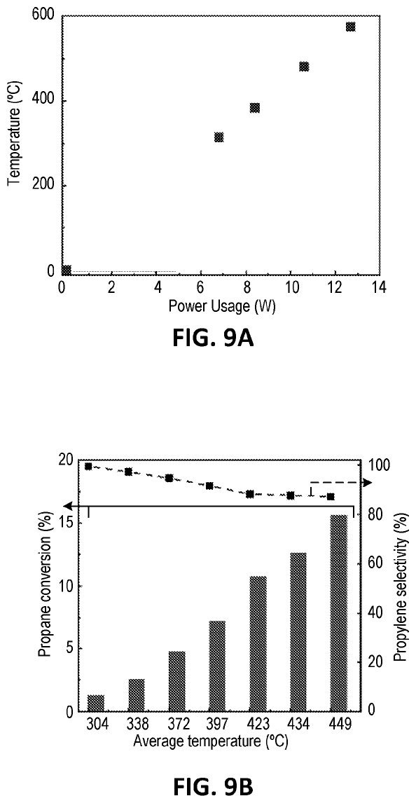

The next step was to load the catalyst and to connect the module with the reactor rig for the catalysis testing. Prior to adding the catalyst, a piece of quartz wool was inserted from the third opening of the Ultra-Torr Tee fitting and then positioned next to one of the copper foil connectors inside the module. The Pt—Zn/S1 catalyst diluted with acid-washed quartz particles was loaded into the reactor from the same opening through which the quartz wool was inserted. The catalyst bed stayed on the top of the quartz wool. The quartz capillary tube that was connected to the CMS hollow fiber was connected to the propane feed gas delivery tunings. The third openings of the tees were connected to the sweep gas delivery tubing. For the electrical circuit, a direct current (DC) power source was directly connected to the Joule heating CMS membrane reactor. The reactor temperature was controlled by the DC power source under constant voltage mode, as shown in FIG. 9A. Temperature along the catalyst bed was recorded in-situ by moving the thermocouple position in the catalyst bed.

Effective Joule heating of the CMS hollow fiber membrane was evidenced by color change as the direct current passed through the membrane. The CMS hollow fiber membrane thus acts as a Joule heating element that heats the catalyst bed for the PDH reaction, without requiring an external heating device (e.g., electric or fuel-powered furnace). The PDH reactions take place in the electrified CMS MR, as shown in FIG. 9B by higher propane conversions at higher catalyst bed temperatures. The electrified CMS reactor is compact, efficient, and amenable to scaling up (as the CMS hollow fibers are heating elements) and can eliminate, or at least reduce use of, the fuel-fired heating required in conventional PDH process.

### Additional Examples of the Disclosed Technology

In view of the above-described implementations of the disclosed subject matter, this application discloses the additional examples in the clauses enumerated below. It should be noted that one feature of a clause in isolation, or more than one feature of the clause taken in combination, and, optionally, in combination with one or more features of one or more further clauses are further examples also falling within the disclosure of this application.

Clause 1. A method comprising:
(a) providing a reactant flow in a first volume of a catalytic membrane reactor, the catalytic membrane reactor comprising at least one hollow fiber membrane and at least one catalyst, each hollow fiber membrane comprising an annular wall formed of carbon, each annular wall separating an interior volume from a surrounding outer volume, the first volume comprising one of (i) the surrounding outer volume and (ii) each interior volume, a second volume of the catalytic membrane reactor comprising the other of (i) the surrounding outer volume and (ii) each interior volume;
(b) at a same time as (a), providing a sweep gas flow in the second volume of the catalytic membrane reactor, or withdrawing permeate from the second volume of the catalytic membrane reactor by applying a vacuum; and
(c) at a same time as (a) and (b), subjecting the catalytic membrane reactor to a temperature in a range of 100-1000° C., inclusive, such that one or more reactants in the reactant flow are converted to one or more products and such that hydrogen permeates through the annular wall and is carried from the second volume by the sweep gas flow or vacuum.

Clause 2. The method of any clause or example herein, in particular, Clause 1, wherein the subjecting of (c) comprises passing an electrical current through each hollow fiber membrane so as to cause Joule heating thereof.

Clause 3. The method of any clause or example herein, in particular, any one of Clauses 1-2, wherein the subjecting of (c) comprises heating the catalytic membrane reactor via:
combustion of gasoline, natural gas, coal, biogas, biomass, or any combination thereof;
focused solar radiation;
electrical heating; or
any combination of the above.

Clause 4. The method of any clause or example herein, in particular, any one of Clauses 1-3, wherein the one or more reactants are converted to one or more products via a direct non-oxidative dehydrogenation reaction.

Clause 5. The method of any clause or example herein, in particular, any one of Clauses 1-4, wherein the one or more reactants comprise an alkane.

Clause 6. The method of any clause or example herein, in particular, any one of Clauses 1-5, wherein the sweep gas flow comprises one or more inert gases.

Clause 7. The method of any clause or example herein, in particular, any one of Clauses 1-6, wherein the one or more reactants comprise one or more hydrocarbons.

Clause 8. The method of any clause or example herein, in particular, any one of Clauses 1-7, wherein:
the one or more reactants comprise propane, and the one or more products comprise propylene; or
the one or more reactants comprise ethane, and the one or more products comprise ethylene; or
the one or more reactants comprise a hydrocarbon, and the one or more products comprise a corresponding olefin.

Clause 9. The method of any clause or example herein, in particular, any one of Clauses 1-8, wherein:
the one or more reactants comprise methane, propane, ethane, n-butane, iso-butane, cyclohexane, hydrogen, nitrogen, carbon monoxide, carbon dioxide, hydrogen sulfide, water, methanol, ethanol, propanol, butanol, or any combination of the foregoing; and/or
the one or more products comprise ethylene, propylene, butylene, butadiene, iso-butylene, benzene, toluene, ethylbenzene, hydrogen, nitrogen, carbon monoxide, carbon dioxide, hydrogen sulfide, ammonia, or any combination of the foregoing.

Clause 10. The method of any clause or example herein, in particular, any one of Clauses 1-9, further comprising, prior to (a):
subjecting at least one precursor hollow fiber to pyrolysis so as to form the at least one hollow fiber membrane, each precursor hollow fiber comprising one or more polymers.

Clause 11. The method of any clause or example herein, in particular, Clause 10, wherein the subjecting to pyrolysis is at a temperature of at least 500° C.

Clause 12. The method of any clause or example herein, in particular, any one of Clauses 10-11, wherein the one or more polymers comprises polysulfones, polyethersulfones, polyetherketones, polyimides, polyetherimides, polyamides, polyamide-imides, polyesters, polybenzimidazoles, polybenzobenzimidazoles, polyethers, or any combination of the foregoing.

Clause 13. The method of any clause or example herein, in particular, any one of Clauses 10-12, wherein each precursor hollow fiber comprises a mixed matrix material comprising metals, metal oxides, zeolites, alumina, silica, metal-organic frameworks, zeolitic imidazolate frameworks, covalent organic frameworks, graphene, graphene oxide, Mxenes, carbon nanotubes, or any combination of the foregoing.

Clause 14. The method of any clause or example herein, in particular, any one of Clauses 1-13, further comprising, prior to (a), forming the at least one hollow fiber membrane by:
exposing at least one precursor hollow fiber to a silane; and
subjecting the at least one precursor hollow fiber to pyrolysis, so as to form the at least one hollow fiber membrane with first and second annular regions,
wherein, after the exposing and the subjecting to pyrolysis, a porosity of the first annular region is greater than a porosity of the second annular region, and/or a density of the first annular region is less than a density of the second annular region.

Clause 15. The method of any clause or example herein, in particular, any one of Clauses 1-14, wherein:
the at least one hollow fiber membrane comprises first and second annular regions, and
a porosity of the first annular region is greater than a porosity of the second annular region, and/or a density of the first annular region is less than a density of the second annular region.

Clause 16. The method of any clause or example herein, in particular, any one of Clauses 14-15, wherein the first annular region is disposed adjacent to the second volume, and the second annular region is disposed adjacent to the first volume.

Clause 17. The method of any clause or example herein, in particular, any one of Clauses 14-16, wherein a thickness of the second annular region is less than a thickness of the first annular region.

Clause 18. The method of any clause or example herein, in particular, any one of Clauses 14-17, wherein a thickness of the second annular region is no more than 10% of a thickness of the first annular region.

Clause 19. The method of any clause or example herein, in particular, any one of Clauses 14-18, wherein a thickness of the second annular region is less than or equal to 5 μum.

Clause 20. The method of any clause or example herein, in particular, any one of Clauses 1-19, wherein an outer diameter of each hollow fiber membrane is less than or equal to 500 μm, and/or an inner diameter of each hollow fiber membrane is less than or equal to 200 μm.

Clause 21. The method of any clause or example herein, in particular, any one of Clauses 1-20, wherein the catalytic membrane reactor comprises a plurality of the hollow fiber membranes.

Clause 22. The method of any clause or example herein, in particular, any one of Clauses 1-21, wherein the first volume comprises the surrounding outer volume, and the second volume comprises the interior volume in each hollow fiber membrane.

Clause 23. The method of any clause or example herein, in particular, any one of Clauses 1-22, wherein the catalytic membrane reactor is configured as a moving bed membrane reactor, a fixed bed membrane reactor, a fluidized bed membrane reactor, or a packed bed membrane reactor.

Clause 24. The method of any clause or example herein, in particular, any one of Clauses 1-23, wherein the at least one catalyst comprises a support without any Lewis or Brønsted acidic centers.

Clause 25. The method of any clause or example herein, in particular, any one of Clauses 1, wherein the at least one catalyst comprises a dehydrogenation catalyst.

Clause 26. The method of any clause or example herein, in particular, Clause 25, wherein the dehydrogenation catalyst comprises single atoms of metal, sub-nanometer-size clusters of metal, nanometer-size clusters of metal, or any combination of the foregoing confined in a siliceous zeolite.

Clause 27. The method of any clause or example herein, in particular, any one of Clauses 25-26, wherein the metal of the dehydrogenation catalyst comprises Pt, Zn, Sn, Cu, Co, Pd, Fe, Ni, Ru, Rh, Ir, Mn, Y, Bi, Zr, Cr, Cs, Li, Na, K, Ca, Ba, or any combination of the foregoing.

Clause 28. The method of any clause or example herein, in particular, any one of Clauses 25-27, wherein a concentration of metal in the dehydrogenation catalyst is in a range of 0.001 wt% to 30 wt%, inclusive.

Clause 29. The method of any clause or example herein, in particular, any one of Clauses 26-28, wherein the siliceous zeolite comprises MFI zeolite, MWW zeolite, FER zeolite, FAU zeolite, BEA zeolite, or MOR zeolite.

Clause 30. The method of any clause or example herein, in particular, any one of Clauses 25-29, further comprising, prior to (a), forming the dehydrogenation catalyst by:
hydrothermal synthesis in an aqueous, sol-gel, or dry state;
dealumination followed by metal precursor impregnation;
in-situ reduction; or
any combination of the above.

Clause 31. The method of any clause or example herein, in particular, any one of Clauses 1-30, wherein the one or more reactants comprise propane, and the one or more products comprise propylene.

Clause 32. The method of any clause or example herein, in particular, any one of Clauses 1-31, wherein:
the sweep flow comprises an inert gas;
the at least one catalyst comprises silicalite-1 supported platinum and zinc (Pt—Zn/S1);
the subjecting is at a temperature of about 450° C.;
at least 30% of propane in the reactant flow is converted into the one or more products after (b);
at least 95% of the one or more products is propylene; or
any combination of the above.

Clause 33. The method of any clause or example herein, in particular, any one of Clauses 1-32, wherein the one or more reactants comprise ethane, and the one or more products comprise ethylene.

Clause 34. The method of any clause or example herein, in particular, any one of Clauses 1-33, wherein:
the sweep flow comprises an inert gas;
the at least one catalyst comprises cobalt in dealuminated beta zeolite (Co/deAl-BEA);
the subjecting is at a temperature of about 500° C.;
at least 12% of ethane in the reactant flow is converted into the one or more products after (b);
at least 80% of the one or more products is ethylene; or
any combination of the above.

Clause 35. The method of any clause or example herein, in particular, any one of Clauses 1-34, wherein the temperature in the subjecting of (c) is less than or equal to 600° C.

Clause 36. The method of any clause or example herein, in particular, any one of Clauses 1-35, wherein the temperature in the subjecting of (c) is in a range of 300-600° C., inclusive.

Clause 37. The method of any clause or example herein, in particular, any one of Clauses 1-36, wherein at least one catalyst is disposed in or adjacent to the first volume.

Clause 38. The method of any clause or example herein, in particular, any one of Clauses 1-37, wherein at least one catalyst is provided within or supported by a porous region of the at least one hollow fiber membrane.

Clause 39. A system comprising:
a catalytic membrane reactor comprising:
   at least one hollow fiber membrane, each hollow fiber membrane comprising an annular wall formed of carbon, each annular wall separating an interior volume from a surrounding outer volume; and
   at least one catalyst,
wherein a reactant flow volume comprises one of (i) the surrounding outer volume and (ii) each interior volume, and a sweep flow volume comprises the other of (i) the surrounding outer volume and (ii) each interior volume, and
the catalytic membrane reactor is configured such that a reactant flow is provided to the at least one catalyst via the reactant flow volume, a product flow exits from the reactant flow volume, and a permeate flow exits from the sweep flow volume.

Clause 40. The system of any clause or example herein, in particular, Clause 39, wherein the catalytic membrane reactor comprises a plurality of the hollow fiber membranes.

Clause 41. The system of any clause or example herein, in particular, any one of Clauses 39-40, wherein the reactant flow volume comprises the surrounding outer volume, and the sweep flow volume comprises the interior volume in each hollow fiber membrane.

Clause 42. The system of any clause or example herein, in particular, any one of Clauses 39-41, wherein the catalytic membrane reactor is configured as a moving bed membrane reactor, a fixed bed membrane reactor, a fluidized bed membrane reactor, or a packed bed membrane reactor.

Clause 43. The system of any clause or example herein, in particular, any one of Clauses 39-42, wherein at least one catalyst is disposed in or adjacent to the reactant flow volume.

Clause 44. The system of any clause or example herein, in particular, any one of Clauses 39-43, wherein at least one catalyst is provided within or supported by a porous region of the at least one hollow fiber membrane.

Clause 45. The system of any clause or example herein, in particular, any one of Clauses 39-44, wherein the at least one catalyst comprises a support without any Lewis or Brønsted acidic centers.

Clause 46. The system of any clause or example herein, in particular, any one of Clauses 39-44, wherein the at least one catalyst comprises a dehydrogenation catalyst.

Clause 47. The system of any clause or example herein, in particular, Clause 46, wherein the dehydrogenation catalyst comprises single atoms of metal, sub-nanometer-size clusters of metal, nanometer-size clusters of metal, or any combination of the foregoing confined in a siliceous zeolite.

Clause 48. The system of any clause or example herein, in particular, any one of Clauses 46-47, wherein the metal of the dehydrogenation catalyst comprises Pt, Zn, Sn, Cu, Co, Pd, Fe, Ni, Ru, Rh, Ir, Mn, Y, Bi, Zr, Cr, Cs, Li, Na, K, Ca, Ba, or any combination of the foregoing.

Clause 49. The system of any clause or example herein, in particular, any one of Clauses 47-48, wherein the siliceous zeolite comprises an MFI zeolite, an MWW zeolite, an FER zeolite, an FAU zeolite, a BEA zeolite, or an MOR zeolite.

Clause 50. The system of any clause or example herein, in particular, any one of Clauses 46-49, wherein a concentration of metal in the dehydrogenation catalyst is in a range of 0.001 wt % to 30 wt %, inclusive.

Clause 51. The system of any clause or example herein, in particular, any one of Clauses 39-50, wherein:
the annular wall of the at least one hollow fiber membrane comprises first and second annular regions, and/or
a porosity of the first annular region is greater than a porosity of the second annular region, and/or a density of the first annular region is less than a density of the second annular region.

Clause 52. The system of any clause or example herein, in particular, Clause 49, wherein the first annular region is disposed adjacent to the sweep volume, and the second annular region is disposed adjacent to the reactant flow volume.

Clause 53. The system of any clause or example herein, in particular, any one of Clauses 51-52, wherein a thickness of the second annular region is less than a thickness of the first annular region.

Clause 54. The system of any clause or example herein, in particular, any one of Clauses 51-53, wherein a thickness of the second annular region is no more than 10% of a thickness of the first annular region.

Clause 55. The system of any clause or example herein, in particular, any one of Clauses 51-54, wherein a thickness of the second annular region is less than or equal to 5 μm.

Clause 56. The system of any clause or example herein, in particular, any one of Clauses 51-55, wherein an outer diameter of each hollow fiber membrane is less than or equal to 500 μm, and/or an inner diameter of each hollow fiber membrane is less than or equal to 200 μm.

Clause 57. The system of any clause or example herein, in particular, any one of Clauses 39-56, further comprising:
a furnace having at least a part of the catalytic membrane reactor disposed therein; and
a controller operatively coupled to the furnace, the controller comprising one or more processors and one or more non-transitory computer-readable media storing computer-readable instructions that, when executed by the one or more processors, cause the one or more processors to control the furnace to heat the at least a part of the catalytic membrane reactor to a temperature in a range of 100-1000° C., inclusive.

Clause 58. The system of any clause or example herein, in particular, Clause 57, wherein the furnace is constructed to provide heating via:
combustion of gasoline, natural gas, coal, biogas, biomass, or any combination thereof;
focused solar radiation;
electrical heating; or
any combination of the above.

Clause 59. The system of any clause or example herein, in particular, any one of Clauses 39-58, further comprising:
a power source electrically coupled to and configured to supply an electrical current to the at least one hollow fiber membrane; and
a controller operatively coupled to the power source, the controller comprising one or more processors and one or more non-transitory computer-readable media storing computer-readable instructions that, when executed by the one or more processors, cause the one or more processors to control the power source to supply the electrical current to the at least one hollow fiber membrane, so as to cause Joule heating of the at least one hollow fiber membrane to a temperature in a range of 100-1000° C.

Clause 60. The system of any clause or example herein, in particular, any one of Clauses 57-59, wherein the temperature is at least 300° C.

Clause 61. The system of any clause or example herein, in particular, any one of Clauses 57-60, wherein the temperature is at least 600° C.

Clause 62. The system of any clause or example herein, in particular, any one of Clauses 39-61, wherein the catalytic membrane reactor is configured to perform a direct non-oxidative dehydrogenation reaction.

Clause 63. The system of any clause or example herein, in particular, any one of Clauses 39-62, further comprising:
a reactant gas source fluidically coupled to the reactant flow volume and configured to provide the reactant flow;
a sweep gas source fluidically coupled to the sweep flow volume and configured to provide the permeate flow;
a first vacuum source fluidically coupled to the reactant flow volume and configured to provide the reactant flow;
a second vacuum source fluidically coupled to the sweep flow volume and configured to provide the permeate flow; or
any combination of the foregoing.

Clause 64. The system of any clause or example herein, in particular, Clause 63, wherein the reactant flow from the reactant gas source comprises one or more light alkanes as the reactant flow, and/or the sweep flow from the sweep gas source comprises one or more inert gases.

Clause 65. A method comprising:
providing the system of any clause or example herein, in particular, any one of Clauses 39-64; and
heating one or more reactants flowing in the catalytic membrane reactor so as to convert the one or more reactants to one or more products.

### CONCLUSION

Any of the features illustrated or described herein, for example, with respect to FIGS. 1A-9B and Clauses 1-65, can be combined with any other feature illustrated or described herein, for example, with respect to FIGS. 1A-9B and Clauses 1-65 to provide materials, systems, devices, structures, methods, and embodiments not otherwise illustrated or specifically described herein. All features described herein are independent of one another and, except where structurally impossible, can be used in combination with any other feature described herein. In view of the many possible embodiments to which the principles of the disclosed technology may be applied, it should be recognized that the illustrated embodiments are only examples and should not be taken as limiting the scope of the disclosed technology. Rather, the scope is defined by the following claims. We therefore claim all that comes within the scope and spirit of these claims.

1. A method comprising:
(a) providing a reactant flow in a first volume of a catalytic membrane reactor, the catalytic membrane reactor comprising at least one hollow fiber membrane and at least one catalyst, each hollow fiber membrane comprising an annular wall formed of carbon, each annular wall separating an interior volume from a surrounding outer volume, the first volume comprising one of (i) the surrounding outer volume and (ii) each interior volume, a second volume of the catalytic membrane reactor comprising the other of (i) the surrounding outer volume and (ii) each interior volume;
(b) at a same time as (a), providing a sweep gas flow in the second volume of the catalytic membrane reactor, or withdrawing permeate from the second volume of the catalytic membrane reactor by applying a vacuum; and
(c) at a same time as (a) and (b), subjecting the catalytic membrane reactor to a temperature in a range of 100-1000° C., inclusive, such that one or more reactants in the reactant flow are converted to one or more products and such that hydrogen permeates through the annular wall and is carried from the second volume by the sweep gas flow or vacuum.

2. The method of claim 1, wherein the subjecting of (c) comprises passing an electrical current through each hollow fiber membrane so as to cause Joule heating thereof.

3. The method of claim 1, wherein the subjecting of (c) comprises heating the catalytic membrane reactor via:
combustion of gasoline, natural gas, coal, biogas, biomass, or any combination thereof;
focused solar radiation;
electrical heating; or
any combination of the above.

4. The method of claim 1, wherein the one or more reactants are converted to one or more products via a direct non-oxidative dehydrogenation reaction.

5. The method of claim 4, wherein the one or more reactants comprise an alkane, and/or the sweep gas flow comprises one or more inert gases.

6-8. (canceled)

9. The method of claim 1, further comprising, prior to (a):
subjecting at least one precursor hollow fiber to pyrolysis so as to form the at least one hollow fiber membrane, each precursor hollow fiber comprising one or more polymers.

10-11. (canceled)

12. The method of claim 9, wherein each precursor hollow fiber comprises a mixed matrix material comprising metals, metal oxides, zeolites, alumina, silica, metal-organic frameworks, zeolitic imidazolate frameworks, covalent organic frameworks, graphene, graphene oxide, Mxenes, carbon nanotubes, or any combination of the foregoing.

13. The method of claim 1, further comprising, prior to (a), forming the at least one hollow fiber membrane by:
exposing at least one precursor hollow fiber to a silane; and
subjecting the at least one precursor hollow fiber to pyrolysis, so as to form the at least one hollow fiber membrane with first and second annular regions,
wherein, after the exposing and the subjecting to pyrolysis, a porosity of the first annular region is greater than a porosity of the second annular region, and/or a density of the first annular region is less than a density of the second annular region.

14. The method of claim 1, wherein:
the at least one hollow fiber membrane comprises first and second annular regions, and
a porosity of the first annular region is greater than a porosity of the second annular region, and/or a density of the first annular region is less than a density of the second annular region.

15-24. (canceled)

25. The method of claim 1, wherein the at least one catalyst comprises a dehydrogenation catalyst, and the dehydrogenation catalyst comprises single atoms of metal, sub-nanometer-size clusters of metal, nanometer-size clusters of metal, or any combination of the foregoing confined in a siliceous zeolite.

26. (canceled)

27. The method of claim 25, wherein a concentration of metal in the dehydrogenation catalyst is in a range of 0.001 wt % to 30 wt %, inclusive.

28. The method of claim 25, wherein the siliceous zeolite comprises MFI zeolite, MWW zeolite, FER zeolite, FAU zeolite, BEA zeolite, or MOR zeolite.

29-30. (canceled)

31. The method of claim 1, wherein the one or more reactants comprise propane, the one or more products comprise propylene, and:
the sweep flow comprises an inert gas;
the at least one catalyst comprises silicalite-1 supported platinum and zinc (Pt—Zn/S1);
the subjecting is at a temperature of about 450° C.;
at least 30% of propane in the reactant flow is converted into the one or more products after (b);
at least 95% of the one or more products is propylene; or
any combination of the above.

32. (canceled)

33. The method of claim 1, wherein the one or more reactants comprise ethane, the one or more products comprise ethylene, and:
the sweep flow comprises an inert gas;
the at least one catalyst comprises cobalt in dealuminated beta zeolite (Co/deAl-BEA);
the subjecting is at a temperature of about 500° C.;
at least 12% of ethane in the reactant flow is converted into the one or more products after (b);
at least 80% of the one or more products is ethylene; or
any combination of the above.

34-36. (canceled)

37. A system comprising:
a catalytic membrane reactor comprising:
at least one hollow fiber membrane, each hollow fiber membrane comprising an annular wall formed of carbon, each annular wall separating an interior volume from a surrounding outer volume; and
at least one catalyst,
wherein a reactant flow volume comprises one of (i) the surrounding outer volume and (ii) each interior volume, and a sweep flow volume comprises the other of (i) the surrounding outer volume and (ii) each interior volume, and
the catalytic membrane reactor is configured such that a reactant flow is provided to the at least one catalyst via the reactant flow volume, a product flow exits from the reactant flow volume, and a permeate flow exits from the sweep flow volume.

38-44. (canceled)

45. The system of claim 37, wherein the at least one catalyst comprises a dehydrogenation catalyst, and the dehydrogenation catalyst comprises single atoms of metal, sub-nanometer-size clusters of metal, nanometer-size clusters of metal, or any combination of the foregoing confined in a siliceous zeolite.

46-48. (canceled)

49. The system of claim 37, wherein:
the annular wall of the at least one hollow fiber membrane comprises first and second annular regions, and
a porosity of the first annular region is greater than a porosity of the second annular region, and/or a density of the first annular region is less than a density of the second annular region.

50-54. (canceled)

55. The system of claim 37, further comprising:
a furnace having at least a part of the catalytic membrane reactor disposed therein; and
a controller operatively coupled to the furnace, the controller comprising one or more processors and one or more non-transitory computer-readable media storing computer-readable instructions that, when executed by the one or more processors, cause the one or more processors to control the furnace to heat the at least a part of the catalytic membrane reactor to a temperature in a range of 100-1000° C., inclusive.

56. The system of claim 55, wherein the furnace is constructed to provide heating via:
combustion of gasoline, natural gas, coal, biogas, biomass, or any combination thereof;
focused solar radiation;
electrical heating; or
any combination of the above.

57. The system of claim 37, further comprising:
a power source electrically coupled to and configured to supply an electrical current to the at least one hollow fiber membrane; and
a controller operatively coupled to the power source, the controller comprising one or more processors and one or more non-transitory computer-readable media storing computer-readable instructions that, when executed by the one or more processors, cause the one or more processors to control the power source to supply the electrical current to the at least one hollow fiber membrane, so as to cause Joule heating of the at least one hollow fiber membrane to a temperature in a range of 100-1000° C.

58-62. (canceled)
